SEQUENCE LISTING (8 sequences)

SEQ ID NO: 1 - DNA, 20 bp, Homo sapiens
gcatccgtactcccaaaatc

SEQ ID NO: 2 - DNA, 20 bp, Homo sapiens
cttcttcatgacctcgccgt

SEQ ID NO: 3 - DNA, 22 bp, Bos taurus
gatcataggactgtattagtgc

SEQ ID NO: 4 - DNA, 20 bp, Bos taurus
ctgacttagagacgaacttg

SEQ ID NO: 5 - DNA, 33 bp, Homo sapiens
gctccgcccgcagtgggatccatgaccgccgcc

SEQ ID NO: 6 - DNA, 30 bp, Homo sapiens
ggatccggatcctcatgccatgtctccgta

SEQ ID NO: 7 - DNA, 22 bp, Rattus norvegicus
ctccagtctgcagaaggtattc

SEQ ID NO: 8 - DNA, 20 bp, Rattus norvegicus
caaccgcaagattggagtgt

(19) United States
(12) Patent Application Publication
(10) Pub. No.: US 2003/0153524 A1
(43) Pub. Date: Aug. 14, 2003

(54) Methods for inhibiting ocular processes

(76) Inventors: David R. Hinton, Venice, CA (US); Shikun He, Temple City, CA (US); Noelynn A. Oliver, Los Altos, CA (US)

Correspondence Address: FIBROGEN, INC., Intellectual Property Department, 225 Gateway Boulevard, South San Francisco, CA, 94080, US

(21) Appl. No.: 10/317,390

(22) Filed: Dec. 11, 2002

Related U.S. Application Data
(60) Provisional application No. 60/339,547, filed on Dec. 11, 2001.

(51) Int. Cl. A61K 48/00; A61K 31/00; A61K 39/395

(52) U.S. Cl. 514/44; 424/145.1; 514/1

(57) ABSTRACT

The present invention relates to methods for inhibiting or preventing ocular processes associated with CTGF. Methods and agents for use in treating or preventing ocular disorders, methods for diagnosing ocular disorders and related kits, and methods for screening for agents for use in the present methods are also provided.

This application claims the benefit of U.S. Provisional Application Serial No. 60/339,547, filed Dec. 11, 2001, which is incorporated by reference herein in its entirety.

### FIELD OF THE INVENTION

The present invention relates generally to methods for inhibiting or preventing ocular processes, including extracellular matrix production and/or deposition, neovascularization, inflammation, cell proliferation, cell migration, etc., in the eye, and to methods and agents for use in preventing and treating ocular disorders.

### BACKGROUND OF THE INVENTION

The eye is a complex organ, and possesses multiple functional layers containing various types of structures, including highly specialized cell types. The retina is the light-sensitive layer lining the back of the eye. The macula is the portion of the retina lying on the optical axis of the eye. The macula provides detailed vision and distinguishes fine details at the center of the field of vision. In a healthy eye, the outer retina is nourished and maintained by an adjoining layer called the retinal pigment epithelium, a monolayer of retinal pigment epithelial (RPE) cells located beneath the retina. Behind the retinal pigment epithelium is the choroid, a highly vascular region containing blood vessels that transport nourishment to and carry waste material away from the retina. Other important structures of the eye include the lens, iris, cornea, trabecular meshwork, and vitreous.

An ocular disorder is any alteration in normal physiology and function of the eye. Ocular disorders can result from a wide range of acute or chronic conditions and events, including injury, insult, or trauma; disease; surgery; etc. Ocular disorders can lead to reduced optical clarity, vision loss, and blindness, seriously compromising quality of life.

The vast majority of ocular disorders are associated with certain physiological processes. Cell proliferation, cell migration, inflammation, neovascularization, and fibrosis (excess production and deposition of extracellular matrix components) develop in response to or in association with, or lead to, changes in ocular morphology and function, and any number of ocular diseases and conditions. Any wound healing response in the eye often includes one or more of these processes.

Examples of ocular disorders, and the ocular processes common to them, include the following:

Proliferative Vitreoretinopathy

Proliferative vitreoretinopathy (PVR) is an ocular disorder resulting from any ocular injury or trauma, including, for example, perforating injury, rhegmatogenous retinal detachment, penetrating injury, intraocular foreign body, contusion, etc. (Cardillo et al. (1997) Ophthalmology 104:1166-1173.) PVR is the most common cause of treatment failure in rhegmatogenous retinal detachment, associated with retinal distortion, breaks, and recurrent detachment. (Girard et al. (1994) Retina 14:417-424.) PVR is associated with cellular proliferation and formation, growth, and contraction of cellular and fibrotic membranes within the vitreous cavity (vitreous membranes) and on both the inner and outer surfaces of the retina (epiretinal membranes). RPE cells proliferation and migration is a major feature of this disorder.

Epiretinal and vitreous membranes contain predominantly RPE cells, but can also contain fibroblasts, glial cells (e.g., Müller cells, astrocytes, microglia, perivascular glia, etc.), and inflammatory cells (e.g., macrophages, lymphocytes, etc.). In addition to cellular components, epiretinal and vitreous membranes, such as the membranes associated with PVR, contain extracellular matrix components, such as collagens (e.g., collagen types I, II, and IV) and fibronectin, and can also contain heparan sulphate, laminin, vitronectin, thrombospondin, etc. The epiretinal and vitreous membranes associated with PVR become progressively paucicellular and fibrotic over time. (Hiscott et al. (1985) Br J Ophthalmol 11:810-823.) PVR and other conditions associated with development of or alterations in ocular membranes can occur as complications of a number of diseases affecting the eye, such as, for example, diabetes.

Macular Degeneration

Macular degeneration, including age-related macular degeneration, is a leading cause of decreased visual acuity and impairment of reading and fine close-up vision. Generally, macular degeneration is characterized by degeneration of the central portion of the retina, known as the macula, resulting in loss of central vision in the affected eye. Degeneration of the macula originates, at least in part, in the retinal pigment epithelium. Macular degeneration is associated with abnormal deposition of extracellular matrix in the basement membrane (Bruch's membrane) found between the retinal pigment epithelium and the vascular choroid. The deposits of extracellular matrix in macular degeneration are called drusen. Two major forms of macular degeneration have been characterized: dry form of macular degeneration, also known as non-exudative, involutional, atrophic macular degeneration or geographic atrophy; and wet form of macular degeneration, also known as exudative, neovascular, or disciform macular degeneration.

Choroidal Neovascularization

Choroidal neovascularization (CNV) is a serious condition, often associated with macular degeneration involving increased choroidal endothelial cell (CEC) adhesion, migration, and proliferation; extracellular matrix overproduction; and development of a fibrovascular subretinal membrane. (D'Amore (1994) IOVS 35:3974-3979; Hinton et al. (1998) Arch Ophthalmol 116:203-209.) Evidence suggests that the retinal pigment epithelium plays a key role in the development of CNV, and that RPE cell dedifferentiation, proliferation, migration, and production of angiogenic factors modulate CNV development. (Grossniklaus et al. (1992) Am J Ophthamol 114:464-472; Das et al. (1992) Ophthamology 99:1368-1376.) While CNV most commonly occurs in macular degeneration, it has been identified as a complication of over forty other ocular disorders.

Wound Healing

Wound healing in response to acute or chronic injury, trauma, disease, etc., can become prolonged or dysregulated, thus leading to more severe ocular tissue damage and loss of vision. The wound healing cascade can include, e.g., initiation of an inflammatory response, recruitment and activation of inflammatory cells, release of growth factors and cytokines, activation, proliferation, migration, and differentiation of various ocular cells, increased capillary permeability, alterations in basement membrane matrix composition, and increased secretion and deposition of extracellular matrix by various ocular cells, fibrosis, neovascularization, tissue remodeling, and tissue repair. (Cordeiro et al. (1999) Br J Ophthalmol 83:1219-1224.)

As the above discussion illustrates, a wide range of ocular disorders are interrelated and encompass common processes. The conditions described above, and other, including, for example, diabetic retinopathy and glaucoma, exemplify the association of serious ocular conditions with these common ocular processes, e.g., ocular neovascularization and ocular scar formation (i.e., ocular fibrosis).

Despite the involvement of common physiological processes such as those described above in the vast majority of ocular disorders, current therapies fail to address these universal mechanisms of ocular disease, and can thus be limited in application. Specifically, no current therapies are directed to treatment of processes such as ocular fibrosis, scar formation, and ocular membrane formation, e.g., epiretinal membrane formation, common to a large number of ocular disorders.

Available treatments for diabetic retinopathy, for example, have been somewhat efficacious in addressing regression of new blood vessel formation and prevention of vitreous hemorrhage and tractional retinal detachments. However, this therapeutic approach does not address, and thus fails to prevent or minimize, the development and progression of various processes (e.g., cell proliferation, cell migration, ocular fibrosis, etc.) that, unchecked, can lead to vision impaired in varying degrees of severity, and even blindness. Current treatments PVR include vitrectomy, involving the removal of vitreous and membranes from the surface of retina; however, ocular inflammation is induced as a result of such treatment, leading to reactivation of pathological processes associated with PVR. In treatment for macular degeneration associated with CNV, photodynamic/laser photocoagulation therapy for occlusion of activated choroidal vessels does not address associated ocular fibrosis, and, in fact, can even induce a wound healing response, that can lead to further complications. Many other current treatments for ocular disorders often apply mechanical means, such as surgery, including laser surgery, to remove or ablate membranes or abnormal ocular tissue, rather than being directed to growth factors and other pathological molecules which mediate ocular processes.

In summary, no current treatments are successfully directed to prevent or inhibit further development and progression in ocular diseases with processes including, e.g., neovascularization, fibrosis, cell proliferation, cell migration, inflammation, etc. There is thus a need for a therapeutic approach that can be broadly applied to treatment of a number of ocular disorders, regardless of origin or development. Further, there is a need for a therapeutic approach that addresses ocular processes universal to the development and progression of ocular disease. In particular, there is a need for therapies that target the fibrotic process and ocular scarring extremely prevalent in macular degeneration, diabetic retinopathy, cataract, and corneal fibrosis, including corneal scarring or hazing, and glaucoma.

Until the present invention, the role of connective tissue growth factor (CTGF) in various ocular processes and associated disorders has remained unclear. CTGF expression in cultured vascular retinal endothelial cells, lens epithelial cells, and rabbit corneal fibroblasts has been reported. (See, e.g., Suzuma et al. (2000) J Biol Chem 275:40725-40731; Lee and Joo (1999) Invest Ophthalmol Vis Sci 40:2025-2032; Park et al. (2001) Biochem Biophys Res Commun 284:966-971; Folger et al. (2001) Invest Ophthalmol Vis Sci 42:2534-2541.) CTGF mRNA expression was observed in corneal scar tissue and retrocorneal membranes, in human anterior subcapsular cataracts and membranes of posterior capsule opacification, and in epiretinal and subretinal fibrovascular membranes. (Wunderlich et al. (2000) Ophthalmologica 214:341-346; Wunderlich et al. (2000) Curr Eye Res 21:627-636; Meyer et al. (2002) Ophthalmologica 216:284-291.) These preliminary observations suggest a need in the art exists for a more complete understanding and characterization of the role of CTGF in ocular disorders.

In summary, there is a need for therapeutic approaches that can be applied to the prevention or treatment of ocular disease and the various physiological processes that can develop in response to or in association with these conditions. There is a further need for an understanding of the role of CTGF in ocular disorders in general, and in the physiological pathways common to many of these disorders. The present invention answers these needs by identifying the role of CTGF in various processes associated with the development and progression of ocular disease, and by providing methods for inhibiting and preventing these processes. The invention further addresses existing needs by providing methods and agents that can be applied to the treatment, pretreatment, and prevention of ocular diseases and associated conditions.

### SUMMARY OF THE INVENTION

The present invention relates to methods for inhibiting or preventing ocular processes. Methods and agents for use in treating or preventing ocular disorders, methods for diagnosing ocular disorders and related kits, and methods for screening for agents for use in the present methods are also provided.

In one embodiment, the present invention provides a method for inhibiting or preventing an ocular process associated with CTGF in a subject, the method comprising administering an agent that decreases expression or activity of CTGF or fragments thereof. These methods can be applied in vivo or in vitro. In various embodiments, the subject is a cell or an animal, preferably a mammal, and most preferably a human.

In one aspect, the agent is selected from the group consisting of an antibody, an antisense oligonucleotide, and a small molecule. In another aspect, the agent is delivered to the eye. Methods in which the agent is delivered to the ocular surface are specifically contemplated. In a preferred aspect, the agent is delivered in an eye drop formulation.

In preferred embodiments, the ocular process is further associated with an ocular cell. In certain embodiments, the ocular cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Müller cell, a perivascular astrocyte, a rod, a cone, a ganglion cell, and a bipolar cell.

In another embodiment, the ocular process is further associated with an ocular structure. The invention provides in various embodiments that the ocular structure is selected from the group consisting of the retina, retinal pigment epithelium layer, choroid, macula, cornea, lens, iris, sclera, trabecular meshwork, aqueous, aqueous chamber, vitreous, ciliary body, optic disc, papilla, and fovea.

Methods for inhibiting or preventing an ocular process associated with CTGF, the method comprising administering to a subject an agent that decreases expression or activity of CTGF or fragments thereof, wherein the ocular process is further associated with an ocular disorder, are specifically contemplated. Application of the present methods to any CTGF-associated ocular disorder is specifically contemplated. In particular embodiments, the ocular disorder is selected from the group consisting of glaucoma, cataract, choroidal neovascularization, retinal detachment, proliferative vitreoretinopathy, macular degeneration, diabetic retinopathy, corneal scarring, and corneal haze. In a preferred embodiment, the ocular process is further associated with ocular fibrosis.

A method for inhibiting or preventing ocular extracellular matrix production or deposition, the method comprising administering to a subject an agent that decreases expression or activity of CTGF or fragments thereof, is specifically provided herein. A method for inhibiting or preventing ocular scarring, the method comprising administering to a subject an agent that decreases expression or activity of CTGF or fragments thereof, is specifically provided. In one embodiment, the present invention provides a method for inhibiting or preventing ocular neovascularization, the method comprising administering to a subject an agent that decreases expression or activity of CTGF or fragments thereof. In further embodiments, the neovascularization is retinal neovascularization, choroidal neovascularization, neovascularization of the iris, or trabecular neovascularization. A method for inhibiting or preventing ocular inflammation, the method comprising administering to a subject an agent that decreases expression or activity of CTGF or fragments thereof, is also provided.

The present invention further contemplates a method for inhibiting or preventing ocular cell proliferation, the method comprising administering to a subject an agent that decreases expression or activity of CTGF or fragments thereof. In various embodiments, the ocular cell proliferation is selected from the group consisting of epithelial cell proliferation, endothelial cell proliferation, retinal pigment epithelial cell proliferation, and choroidal endothelial cell proliferation.

In one aspect, the present invention provides a method for inhibiting or preventing ocular cell migration, the method comprising administering to a subject an agent that decreases expression or activity of CTGF or fragments thereof. In particular aspects, the ocular cell migration is selected from the group consisting of epithelial cell migration, retinal pigment epithelial cell migration, endothelial cell migration, and choroidal endothelial cell migration.

The present invention further provides a method for inhibiting or preventing an ocular process associated with CTGF in a subject, the method comprising administering an agent that decreases expression or activity of CTGF or fragments thereof, wherein the ocular process is further associated with surgery. In various aspects, the surgery is selected from the group consisting of refraction correction surgery, radial keratotomy, LASIK, retinal detachment surgery, corneal transplantation, glaucoma filtration surgery, cataract extraction surgery, lens replacement surgery, vitrectomy, subretinal surgery, and retinal translocation surgery.

Methods for treating or preventing ocular disorders are specifically provided by the present invention. In one embodiment, the present invention provides a method for treating or preventing an ocular disorder, the method comprising administering to a subject an agent that decreases expression or activity of CTGF or fragments thereof. In another embodiment, a method for treating or preventing ocular fibrosis, the method comprising administering to a subject an agent that decreases expression or activity of CTGF or fragments thereof, is specifically contemplated.

In one aspect, the present invention provides a method for treating or preventing an ocular disorder associated with neovascularization, the method comprising administering to a subject an agent that decreases expression or activity of CTGF or fragments thereof. In various aspects, the neovascularization is retinal neovascularization, choroidal neovascularization, neovascularization of the iris, or trabecular neovascularization. In a particular aspect, a method for treating or preventing choroidal neovascularization associated with macular degeneration is specifically contemplated.

A method for treating or preventing an ocular disorder associated with extracellular matrix production or deposition, the method comprising administering to a subject an agent that decreases expression or activity of CTGF or fragments thereof, is further provided, as is a method for treating or preventing an ocular disorder associated with inflammation, the method comprising administering to a subject an agent that decreases expression or activity of CTGF or fragments thereof.

In one embodiment, the present invention contemplates a method for treating or preventing an ocular disorder associated with cell proliferation, the method comprising administering to a subject an agent that decreases expression or activity of CTGF or fragments thereof. In various embodiments, the cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Müller cell, a perivascular astrocyte, a rod, a cone, a ganglion cell, and a bipolar cell.

In another embodiment, a method for treating or preventing an ocular disorder associated with cell migration, the method comprising administering to a subject an agent that decreases expression or activity of CTGF or fragments thereof, is provided. In certain embodiments, the cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Müller cell, a perivascular astrocyte, a rod, a cone, a ganglion cell, and a bipolar cell. In certain aspect, the present invention provides methods for treating or preventing ocular disorders associated with altered ocular cell migration, chemotaxis, proliferation, or attachment.

A method for treating or preventing a disorder associated with formation or alteration of an ocular membrane, the method comprising administering to a subject an agent that decreases expression or activity of CTGF or fragments thereof, is specifically contemplated. In various aspects, the ocular membrane is selected from the group consisting of an epiretinal membrane, a subretinal membrane, a vitreous membrane, a cellular membrane, a choroidal neovascularization membrane, and a fibrotic membrane.

In certain embodiments, the methods for treating or preventing an ocular disorder are administered prior to, at the time of, or in close association with surgery, in order to treat or to prevent the development of ocular disorders that can develop in association with a surgical event. In various aspects, the surgery is selected from the group consisting of refraction correction surgery, radial keratotomy, LASIK, retinal detachment surgery, corneal transplantation, glaucoma filtration surgery, cataract extraction surgery, lens replacement surgery, vitrectomy, subretinal surgery, and retinal translocation surgery.

The present invention provides a method for maintaining or improving vision in a subject, the method comprising administering to the subject an effective amount of an agent that decreases expression or activity of CTGF or fragments thereof.

Methods for diagnosing an ocular disorder are specifically contemplated. In one embodiment, the present invention provides a method for diagnosing an ocular disorder associated with CTGF, the method comprising obtaining a sample from a subject and measuring the level of CTGF or fragments of CTGF in the sample, and comparing the level of CTGF or fragments thereof in the sample to a standard level, wherein an increased amount of CTGF or fragments thereof in the sample is indicative of the presence of an ocular disorder. In another embodiment, the present invention provides a method for identifying a predisposition to develop an ocular disorder associated with CTGF, the method comprising obtaining a sample from a subject and measuring the level of CTGF or fragments of CTGF in the sample, and comparing the level of CTGF or fragments thereof in the sample to a standard level, wherein an increased amount of CTGF or fragments thereof in the sample is indicative of a predisposition to develop an ocular disorder.

The present invention also provides diagnostic kits for use in diagnosing an ocular disorder associated with CTGF. Diagnostic kits of the present invention comprise a means for detecting the level of CTGF in a sample and a means for measuring the level of CTGF in the sample. In one aspect, the diagnostic kit is useful for diagnosing an ocular disorder associated with CTGF. In another aspect, the diagnostic kit is useful for identifying a predisposition to develop an ocular disorder associated with CTGF. In a preferred embodiment, a diagnostic kit of the present invention is useful for diagnosing ocular fibrosis. In another preferred embodiment, a diagnostic kit of the present invention is useful for identifying a predisposition to develop ocular fibrosis.

Methods for identifying an agent useful for inhibiting or preventing an ocular process are also provided by the present invention.

In one embodiment, the present invention provides a method for identifying an agent for use in inhibiting or preventing an ocular process associated with CTGF, the method comprising contacting a candidate agent with CTGF or a fragment thereof, detecting the level of CTGF expression or activity in the sample, and comparing the level of CTGF expression or activity in the sample to a standard level of CTGF expression or activity, wherein decreased expression and activity of CTGF or fragments thereof in the sample is indicative of an agent that inhibits or prevents an ocular process associated with CTGF.

In another embodiment, the present invention provides a method for identifying an agent for use in treating or preventing an ocular disorder associated with CTGF, the method comprising contacting a candidate agent with CTGF or a fragment thereof detecting the level of CTGF expression or activity in the sample, and comparing the level of CTGF expression or activity in the sample to a standard level of CTGF expression or activity, wherein decreased expression and activity of CTGF or fragments thereof in the sample is indicative of an agent that treats or prevents an ocular disorder associated with CTGF.

The present invention provides a method for treating or preventing macular degeneration. Any of the methods described in the present invention can be applied to treat or prevent any form of macular degeneration, including age-related macular degeneration, dry form of macular degeneration, also known as non-exudative, involutional, atrophic macular degeneration, and geographic atrophy when severe, and wet form of macular degeneration, also know as exudative, neovascular, or disciform macular degeneration. In a preferred embodiment, the present invention provides a method for treating or preventing choroidal neovascularization associated with macular degeneration. In another embodiment, the present invention provides a method for treating or preventing diabetic retinopathy, including proliferative diabetic retinopathy and non-proliferative diabetic retinopathy. In a preferred embodiment, the present invention provides a method for treating or preventing proliferative vitreoretinopathy. In another aspect, the present invention provides a method of treating or preventing retinal detachment. In yet another aspect, the present invention provides a method for treating or preventing glaucoma. In still another aspect, the present invention provides a method of treating or preventing cataracts. In one embodiment, the present invention provides a method of treating or preventing corneal scarring. In another embodiment, the present invention provides a method of treating or preventing corneal haze.

These and other embodiments of the subject invention will readily occur to those of skill in the art in light of the disclosure herein, and all such embodiments are specifically contemplated.

### BRIEF DESCRIPTION OF THE DRAWINGS

FIGS. 1A and 1B show expression of connective tissue growth factor mRNA in retinal pigment epithelial cells and in choroidal endothelial cells, respectively.

FIGS. 2A and 2B show connective tissue growth factor expression in retinal pigment epithelial cells after transforming growth factor-β or vascular endothelial cell growth factor treatment.

FIGS. 3A and 3B show connective tissue growth factor expression in choroidal endothelial cells after vascular endothelial cell growth factor or transforming growth factor-β treatment.

FIGS. 4A and 4B show the effects of connective tissue growth factor on attachment of retinal pigment epithelial cells and choroidal endothelial cells to fibronectin.

FIGS. 5A and 5B show the effects of connective tissue growth factor on retinal pigment epithelial cell and choroidal endothelial cell chemotactic migration.

FIGS. 6A and 6B show dose-dependent stimulation of retinal pigment epithelial cell migration by connective tissue growth factor and N-terminal fragments of connective tissue growth factor.

FIG. 7 shows connective tissue growth factor stimulation of retinal pigment epithelial cell proliferation.

FIGS. 8A and 8B show connective tissue growth factor stimulation of choroidal endothelial cell tube formation.

FIG. 9 shows stimulation of extra domain A containing fibronectin expression by connective tissue growth factor in retinal pigment epithelial cells treated with transforming growth factor-β.

FIG. 10 shows fibronectin stimulates expression of connective tissue growth factor in retinal pigment epithelial cells.

FIG. 11 shows inhibition of fibronectin-induced connective tissue growth factor expression in retinal pigment epithelial cells by anti-α₅β₁ integrin antibody.

FIG. 12 shows connective tissue growth factor expression in human proliferative vitreoretinopathy membranes.

FIGS. 13A, 13B, 13C, and 13D show connective tissue growth factor expression in human choroidal neovascularization membranes from patients with age-related macular degeneration.

FIG. 14 shows connective tissue growth factor and fragments of connective tissue growth factor in vitreous fluid excised from individuals with proliferative vitreoretinopathy.

FIG. 15 shows connective tissue growth factor and fragments of connective tissue growth factor in vitreous fluid surgically excised from individuals with choroidal neovascularization and retinal neovascularization.

FIGS. 16A and 16B show connective tissue growth factor induction of fibrosis in an experimental model of proliferative vitreoretinopathy.

FIG. 17 shows connective tissue growth factor expression in vitreous fluid of rabbits with various experimental models of proliferative vitreoretinopathy.

### DETAILED DESCRIPTION OF THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention. Each reference cited herein is incorporated herein by reference in its entirety.

The practice of the present invention will employ, unless otherwise indicated, conventional methods of chemistry, biochemistry, molecular biology, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Colowick et al. Methods In Enzymology, Academic Press, Inc.; Handbook of Experimental Immunology, Vols. I-IV (D. M. Weir and C. C. Blackwell, eds., (1986) Blackwell Scientific Publications); Maniatis et al.(1989) Molecular Cloning: A Laboratory Manual, 2^{nd} edition, Vols. I-III, Cold Spring Harbor Laboratory Press; Ausubel et al.(1999) Short Protocols in Molecular Biology, 4^{th} edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., (1997), Springer Verlag).

Definitions

The terms “ocular disorder” or “ocular disease” are used inclusively and refer to any condition deviating from normal. In particular, the terms “ocular disorder” or “ocular disease” refer to any abnormality of the eye. Ocular disorders can result from any acute or chronic condition or event affecting the eye, including injury, trauma, disease, surgery, etc. Ocular disorders may be associated with various structures in the eye, such as, for example, the retina, macula, vitreous humor, aqueous humor, lens, cornea, sclera, choroid, ciliary body, ins, optic disc, papilla, fovea, or any other portion of the eye. Additionally, ocular disorders can be associated with various ocular cells, such as RPE cells, retinal and choroidal endothelial cells, trabecular meshwork cells, fibroblasts, corneal fibroblasts, glial cells, Müller cells, epithelial cells, such as, for example, lens epithelial cells, corneal epithelial cells, etc.

Ocular disorders or diseases can be associated with any one or more of various ocular processes, including, for example, neovascularization, cell proliferation, cell migration, inflammation, extracellular matrix production and deposition (e.g., fibrosis), etc. Examples of ocular disorders include, e.g., proliferative vitreoretinopathy, non-proliferative diabetic retinopathy, proliferative diabetic retinopathy, diabetic macular edema, dry macular degeneration, wet macular degeneration, age-related macular degeneration, pigment epithelial detachment associated with macular degeneration, Stargardt's Disease (juvenile macular degeneration), glaucoma, including primary open angle and secondary glaucoma, neovascular glaucoma with proliferative diabetic retinopathy, wound healing associated with glaucoma filtering surgery, idiopathic preretinal fibrosis, subretinal fibrosis, uveal disorders, such as, for example, uveitis syndrome and inflammation of the uvea, idiopathic sclerosing inflammation of the orbit, multifocal fibrosclerosis with orbital involvement, cicatricial pemphigoid with ocular manifestations, systemic lupus with central serous chorioretinopathy, Coats disease with hyperplastic primary vitreous, Vogt-Koyanagi-Harada disease, macular edema, retinal detachment and associated complications, including complications due to retinal detachment surgery, cataracts, macular holes, retinal tears, acute retinal necrosis syndrome, traumatic chorioretinopathies, Purtscher's retinopathy (contusion), retinal edema, corneal epithelial wounds, corneal scarring, corneal haze complicating laser-assisted in situ keratomileusis (LASIK), Sjogren's syndrome, corneal neovascularization, iris neovascularization, choroid neovascularization, vitreous neovascularization, ocular neovascularization, ocular inflammation, retinal degenerations, retinal ischemia, vitreoretinal disorders, ocular fibrosis of unknown etiology, sickle cell retinopathy, retinopathy of prematurity, retinal detachment, ocular ischemia, ocular trauma, and radiation retinopathy.

“Fibrosis” is used herein in its broadest sense referring to any excess production or deposition of extracellular matrix proteins. Fibrosis includes any abnormal processing of fibrous tissue, or fibroid or fibrous degeneration. Fibrosis can result from various injuries or diseases, and typically involves the abnormal production, accumulation, or deposition of extracellular matrix components, including overproduction and increased deposition of, for example, collagen and fibronectin. The term “ocular fibrosis” refers to fibrosis affecting the eye or some portion thereof.

“Ocular cells” are cells associated with any structure of the eye, internal or external, including endothelial cells, epithelial cells, fibroblasts, glial cells, retinal pigment epithelial cells, retinal endothelial cells, choroidal endothelial cells, lens epithelial cells, corneal epithelial cells, trabecular meshwork cells, pericytes, astrocytes, microglial cells, perivascular glial cells, Muller cells, perivascular astrocytes, rods, cones, ganglion cells, bipolar cells, etc.

“Connective tissue growth factor” or “CTGF” refers to CTGF derived from any species, preferably a mammalian species, including rat, rabbit, bovine, ovine, porcine, murine, and equine species, and most preferably the human species, and from any source, whether natural, synthetic, semi-synthetic, or recombinant. Connective tissue growth factor has been reported and described previously. (See, e.g., U.S. Pat. No. 5,408,040; Bradham et al. 1991, J. Cell Biology 114:1285-1294.) In one aspect, “connective tissue growth factor” or “CTGF” refers to a polypeptide sequence comprising at least a portion of the N-terminal fragment of CTGF. In another aspect, “connective tissue growth factor” or “CTGF” refers to a polypeptide sequence comprising at least a portion of the C-terminal fragment of CTGF. The term “N-terminal fragment” used in reference to CTGF means any polypeptide comprising sequence derived from the amino-terminal portion of a CTGF polypeptide, or to any variants, or fragments thereof. The term “C-terminal fragment” used in reference to CTGF means any polypeptide comprising sequence derived from the carboxy-terminal portion of a CTGF polypeptide, or to any variants, of fragments thereof. (See, e.g., International Publication No. WO 00/35939; International Publication No. WO 00/35936; and U.S. application Ser. No. 10/245,977.)

The phrase “CTGF-associated ocular disorders” as used herein refers to ocular conditions and diseases associated with abnormal or inappropriate expression or activity of CTGF or fragments thereof. The term specifically contemplates diseases and disorders associated with ocular cell proliferation, ocular cell migration, extracellular matrix production or deposition in the eye, including ocular fibrosis and scarring, diseases and disorders associated with inflammation in the eye, and ocular angiogenesis and neovascularization.

The proliferative processes and disorders referred to herein include pathological states characterized by the continual multiplication of cells resulting in an overgrowth of a cell population within a tissue. The cell populations are not necessarily transformed, tumorigenic, or malignant cells, but can include normal cells as well. For example, CTGF may be involved pathologically by inducing a proliferative response in various cells of the retina, such as retinal pigment epithelial cells, or by stimulating neovascularization.

The terms “angiogenesis” and “neovascularization” refer to the formation or growth of new or existing capillaries or blood vessels.

The term “agonist” as used in reference to CTGF refers to a molecule which increases or prolongs the extent or duration of the effect of the biological or immunological activity or expression of a particular molecule, e.g., CTGF or fragments of CTGF. Agonists may include proteins, nucleic acids, carbohydrates, antibodies, small molecules, or any other molecules which increase the effect of CTGF or fragments thereof.

The term “antagonist” as used in reference to CTGF refers to a molecule which decreases the extent or duration of the effect of the biological or immunological activity or expression of a particular molecule, e.g., CTGF or fragments of CTGF. Antagonists may include proteins, nucleic acids, carbohydrates, antibodies, small molecules, or any other molecules which decrease the effect of CTGF or fragments thereof.

The term “antibody” refers to immunoglobulins or antibodies, monoclonal or polyclonal, obtained from any source. Such sources can include a cell line, or an animal such as mouse, rat, rabbit, chicken, turkey, goat, horse, human, etc. Antibodies may also be obtained from genetically modified cells, or transgenic plants or animals engineered to make antibodies that are not endogenous to the host. An antibody may be of any isotype including, e.g., IgA, IgD, IgE, IgG-1, IgG-2, IgG-3, IgG-4, or IgM, etc. As used herein, “antibody” includes intact molecules and fragments thereof, such as Fab, F(ab′)₂, and Fv fragments, capable of binding the epitopic determinant, chimeric antibodies, e.g., bivalent and trivalent antibodies, that bind to one or more unique antigen(s).

The term “antisense” refers to any composition containing a nucleic acid sequence which is complementary to the “sense” strand of a specific nucleic acid sequence. Antisense molecules may be produced by any method available in the art including by synthesis or transcription. Once introduced into a cell, the complementary nucleotides can combine with natural sequences produced by the cell to form duplexes and to block either transcription or translation of the corresponding natural sequences.

“Amino acid sequence” or “polypeptide” or “polypeptides,” as these terms are used herein, refer to oligopeptide, peptide, polypeptide, or protein sequences, and fragments thereof, and to naturally occurring or synthetic molecules. Polypeptide or amino acid fragments are any portion of a polypeptide which retains at least one structural and/or functional characteristic of the polypeptide. CTGF fragments include any portion of a CTGF polypeptide sequence which retains at least one structural or functional characteristic of CTGF. Where “amino acid sequence” refers to the polypeptide sequence of a naturally occurring protein molecule, “amino acid sequence” and like terms are not meant to limit the amino acid sequence to the complete native sequence associated with the protein molecule in question.

The terms “nucleic acid” or “polynucleotide” or “polynucleotides” refer to oligonucleotides, nucleotide sequences, or polynucleotides, or any fragments thereof, and to DNA or RNA of natural or synthetic origin which may be single- or double-stranded and may represent the sense or antisense strand, to peptide nucleic acid (PNA), or to any DNA-like or RNA-like material, natural or synthetic in origin. Polynucleotide fragments are any portion of a polynucleotide sequence that retains at least one structural or functional characteristic of the polynucleotide. Polynucleotide fragments can be of variable length, for example, greater than 60 nucleotides in length, at least 100 nucleotides in length, at least 1000 nucleotides in length, or at least 10,000 nucleotides in length.

“Altered” polynucleotides include those with deletions, insertions, or substitutions of different nucleotides resulting in a polynucleotide that encodes the same or a functionally equivalent polypeptide. Included within this definition are sequences displaying polymorphisms that may or may not be readily detectable using particular oligonucleotide probes or through deletion of improper or unexpected hybridization to alleles, with a locus other than the normal chromosomal locus for the subject polynucleotide sequence.

“Altered” polypeptides may contain deletions, insertions, or substitutions of amino acid residues which produce a silent change and result in a functionally equivalent polypeptide. Deliberate amino acid substitutions may be made on the basis of similarity in polarity, charge, solubility, hydrophobicity, hydrophilicity, and/or the amphipathic nature of the residues as long as the biological or immunological activity of the encoded polypeptide is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

A polypeptide or amino acid “variant” is an amino acid sequence that is altered by one or more amino acids from a particular amino acid sequence. A polypeptide variant may have conservative changes, wherein a substituted amino acid has similar structural or chemical properties to the amino acid replaced, e.g., replacement of leucine with isoleucine. A variant may also have non-conservative changes, in which the substituted amino acid has physical properties different from those of the replaced amino acid, e.g., replacement of a glycine with a tryptophan. Analogous minor variations may also include amino acid deletions or insertions, or both. Preferably, amino acid variants retain certain structural or functional characteristics of a particular polypeptide. Guidance in determining which amino acid residues may be substituted, inserted, or deleted may be found, for example, using computer programs well known in the art, such as LASERGENE software (DNASTAR Inc., Madison, Wis.).

A polynucleotide variant is a variant of a particular polynucleotide sequence that preferably has at least about 80%, more preferably at least about 90%, and most preferably at least about 95% polynucleotide sequence similarity to the particular polynucleotide sequence. It will be appreciated by those skilled in the art that as a result of the degeneracy of the genetic code, a multitude of variant polynucleotide sequences encoding, a particular protein, some bearing minimal homology to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard codon triplet genetic code, and all such variations are to be considered as being specifically disclosed.

A “deletion” is a change in an amino acid or nucleotide sequence that results in the absence of one or more amino acid residues or nucleotides.

The terms “insertion” or “addition” refer to a change in a polypeptide or polynucleotide sequence resulting in the addition of one or more amino acid residues or nucleotides, respectively, as compared to the naturally occurring molecule.

The term “functional equivalent” as it is used herein refers to a polypeptide or polynucleotide that possesses at least one functional or structural characteristic of a particular polypeptide or polynucleotide. A functional equivalent may contain modifications that enable the performance of a specific function. The term “functional equivalent” is intended to include fragments, mutants, hybrids, variants, analogs, or chemical derivatives of a molecule.

The term “sample” is used herein in its broadest sense. Samples may be derived from any source, for example, from bodily fluids, secretions, tissues, cells, or cells in culture, including, but not limited to, vitreous humour, aqueous humor, tears, saliva, blood, urine, serum, plasma, synovial fluid, cerebral spinal fluid, amniotic fluid, and organ tissue (e.g., biopsied tissue); from chromosomes, organelles, or other membranes isolated from a cell; from genomic DNA, cDNA, RNA, mRNA, etc.; and from cleared cells or tissues, or blots or imprints from such cells or tissues. Samples may be derived from any source, such as, for example, a cell, a human subject, a non-human mammalian subject, etc. Also contemplated are samples derived from any animal model of disease. A sample can be in solution or can be, for example, fixed or bound to a substrate. A sample can refer to any material suitable for testing for the presence of CTGF or fragments of CTGF or suitable for screening for molecules that bind to CTGF or fragments thereof. Methods for obtaining such samples are within the level of skill in the art.

As used herein, the terms “extracellular matrix” and “extracellular matrix proteins” refer broadly to non-cellular matrix, typically composed of proteins, glycoproteins, complex carbohydrates, and other macromolecules. Extracellular matrix proteins include, for example, collagens, such as collagen types I and IV, fibronectin, laminin, and thrombospondin.

The term “microarray” refers to any arrangement of molecules, e.g. nucleic acids, amino acids, antibodies, etc., on a substrate. The substrate can be any suitable support, e.g., beads, glass, paper, nitrocellulose, nylon, or any appropriate membrane, etc. A substrate can be any rigid or semi-rigid support including, but not limited to, membranes, filters, wafers, chips, slides, fibers, beads, including magnetic or nonmagnetic beads, gels, tubing, plates, polymers, microparticles, capillaries, etc. The substrate can provide a surface for coating and/or can have a variety of surface forms, such as wells, pins, trenches, channels, and pores, to which the nucleic acids, amino acids, etc., may be bound.

The terms “proliferative vitreoretinopathy” or “PVR” refer a variety of intraocular pathologies, including epiretinal membranes, subretinal stands, etc., and retinal detachment in combination with star folds, vitreous traction, anterior loop traction, etc. PVR also includes any intraocular cellular proliferation associated with any degree of retinal detachment.

The Invention

The present invention relates to methods for inhibiting or preventing ocular processes associated with, e.g., cell proliferation, cell migration, neovascularization, inflammation, fibrosis, etc. Methods for preventing and treating ocular disorders are also provided.

Acute or chronic conditions and events, such as, for example, injury, trauma, disease, surgery, etc., are associated with development and progression of one or more ocular processes that can lead to various structural, pathological, physiological, and cellular changes within the eye. Such processes include, e.g., alterations in basement membrane matrix composition; increased production and deposition of extracellular matrix leading to fibrosis and scar formation; neovascularization; increased capillary permeability; ocular cell hyperplasia; ocular cell proliferation; ocular cell migration; development of ocular membranes, such as cellular, fibrotic, subretinal, epiretinal, vitreous, choroidal neovascular membranes, etc.

These ocular processes are associated with many ocular disorders and diseases and can often occur in association with each other. The interrelatedness of multiple ocular processes is evident, for example, in PVR, in which RPE cell proliferation and RPE cell migration are associated with the formation of cellular and fibrotic membranes within the vitreous (vitreous membranes) or on the inner or outer surfaces of the retina (epiretinal membranes). Diabetic retinopathy is associated with damage or alterations in the inner blood vessels of the retina, resulting in hemorrhage and swelling, leaking of fluid into the retina, and new growth of blood vessels on the surface of the retina and into the vitreous. CNV is associated with increased choroidal epithelial cell adhesion, migration, and proliferation; extracellular matrix overproduction; and development of a fibrovascular subretinal membrane. It is understood, therefore, that various ocular processes are interrelated, and thus provide therapeutic pathways common to a wide range of disorders and diseases affecting the eye. Effective methods of inhibiting and preventing these processes, and treating and preventing the associated disorders, thus have broad application.

The present invention relates to the discovery that CTGF is a central factor in the initiation, development, and progression of various ocular processes. Further, the present invention demonstrates that expression and activity of CTGF are associated with the onset and extent of ocular disorders in general, and, in particular, with disorders associated with the processes enumerated above.

CTGF in Ocular Processes and in Ocular Disorders

Involvement of CTGF in Ocular Processes and Ocular Disorders

The present invention demonstrates increased expression of CTGF in various ocular processes and associated diseases and conditions. For example, CTGF expression was elevated in vitreous of patients diagnosed with certain ocular diseases, and in animal models of ocular disorders. (See, e.g., Examples 14, 15, and 22.) The level of CTGF was highest in vitreous samples taken from patients with ocular processes commonly associated with wound healing, such as, for example, neovascularization and fibrosis. (See, e.g., Examples 14 and 15.) Increased CTGF expression was observed in vitreous samples obtained from patients with retinal detachment without PVR, retinal detachment with PVR, epiretinal membranes, macular hole, CNV, retinal neovascularization, proliferative diabetic retinopathy, and age-related macular degeneration. (See, e.g., Example 14.)

Additionally, the inventors found CTGF expression is elevated in ocular tissues obtained from individuals having ocular disorders, such as PVR, diabetic retinopathy, and CNV, including CNV associated with macular degeneration. For example, immunohistochemical data show CTGF was expressed in PVR membranes surgically excised from patients with retinal detachment. (See, e.g., Example 12.) Increased expression of CTGF was observed in CNV membranes obtained from subjects with clinically diagnosed age-related macular degeneration. (See, e.g., Example 13.) Within isolated CNV membranes, CTGF was predominantly localized to neovascular membranes as well as associated with cells surrounding blood vessels. CTGF is thus associated with neovascularization, ocular fibrosis, and ocular membrane formation.

The present inventors further demonstrated expression of CTGF in various ocular cells, including, e.g., RPE cells, CECs, and retinal Müller cells. The present invention shows that CTGF expression in various ocular cells is differentially regulated by various growth factors and extracellular matrix components associated with various ocular disorders and ocular processes. Specifically, TGFβ stimulated CTGF expression in RPE cells, and VEGF stimulated CTGF expression in CECs. (See, e.g., Example 2.) Fibronectin, an extracellular matrix component associated with ocular cell migration and proliferation, stimulated CTGF expression in RPE cells. (See, e.g., Example 10.)

CTGF Induces Ocular Fibrosis

The present invention provides the first demonstration that CTGF induces ocular fibrosis (increased extracellular matrix production and deposition). The present inventors found that CTGF induced pathologic ocular fibrosis in vivo in validated animal models of ocular disease and that addition of CTGF to vitreous stimulated development and progression of ocular fibrosis. (See, e.g., Examples 17 and 19.) Ocular fibrosis induced by CTGF was associated with transformation of epiretinal RPE membranes into paucicellular, myofibroblastic, and densely fibrotic epiretinal membranes. CTGF injected into the vitreous of a rabbit with a mild degree of PVR resulted in the formation of densely fibrotic membranes and development and progression of ocular fibrosis. (See, e.g., Examples 17 and 19.) Further, the present inventors showed that CTGF stimulated extracellular matrix production by demonstrating that CTGF induced expression of fibronectin, a component of extracellular matrix, in RPE cells. (See, e.g., Example 9.) These results provide direct evidence that CTGF induces ocular fibrosis, and thus CTGF represents a target for inhibition of the development and progression of the fibrotic process in the eye (i.e., ocular fibrosis) and associated diseases and disorders.

The present invention demonstrates that CTGF stimulated extracellular matrix production, a feature of ocular fibrosis, in ocular cells. Specifically, the present inventors showed that CTGF induced the expression of fibronectin in RPE cells. (See, e.g., Example 9.)

CTGF Induces Ocular Angiogenesis and Ocular Neovascularization

The present inventors also provide evidence that CTGF induces ocular angiogenesis and ocular neovascularization. Neovascularization is associated with various ocular disorders and ocular processes, including, for example, the development and progression of ocular membranes, proliferative neovascularization, and choroidal neovascularization.

Using an in vitro tube formation assay, in which endothelial cells undergo proliferation, migration, and differentiation, the present inventors demonstrated that monolayer cultures of CECs exhibited increased tube formation in response to CTGF, thus establishing the involvement of CTGF in ocular angiogenesis and neovascularization. (See, e.g., Example 8.)

Further findings showed that CTGF was predominantly localized to neovascular membranes as well as associated with cells surrounding blood vessels in CNV membranes. (See, e.g., Example 13.) The present inventors thus were able to establish the involvement of CTGF in ocular angiogenesis and neovascularization, and neovascular membrane formation.

CTGF Stimulates Ocular Cell Proliferation and Migration

In many ocular disorders, proliferation and migration of ocular cells are associated with development and progression of various ocular processes and associated diseases and disorders. The present invention demonstrates that CTGF stimulates proliferation and migration of ocular cells critically involved in the development and progression of ocular processes, such as, for example, cell proliferation, cell migration; neovascularization, and fibrosis. For example, RPE cell proliferation was stimulated by CTGF. (See, e.g., Examples 7.) Additionally, CTGF stimulated migration of RPE cells, CECs, and retinal Müller cells. (See, e.g., Examples 6 and 11.)

Methods for Inhibiting or Preventing Ocular Processes and Ocular Disorders

The present invention relates to methods for inhibiting or preventing ocular processes. Methods and agents for use in treating or preventing ocular disorders, methods for diagnosing ocular disorders and related kits, and methods for screening for agents for use in the present methods are also provided.

In one embodiment, the present invention provides a method for inhibiting or preventing an ocular process associated with CTGF in a subject, the method comprising administering an agent that decreases expression or activity of CTGF or fragments thereof. These methods can be applied in vivo or in vitro. In various embodiments, the subject is a cell or an animal, preferably a mammal, and most preferably a human.

In one aspect, the agent is selected from the group consisting of an antibody, an antisense oligonucleotide, and a small molecule. In another aspect, the agent is delivered to the eye. Methods in which the agent is delivered to the ocular surface are specifically contemplated. In a preferred aspect, the agent is delivered in an eye drop formulation. In another aspect, the agent is delivered as an ointment.

In preferred embodiments, the ocular process is further associated with an ocular cell. In certain embodiments, the ocular cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Müller cell, a perivascular astrocyte, a rod, a cone, a ganglion cell, and a bipolar cell.

In another embodiment, the ocular process is further associated with an ocular structure. The invention provides in various embodiments that the ocular structure is selected from the group consisting of the retina, retinal pigment epithelium layer, choroid, macula, cornea, lens, iris, sclera, trabecular meshwork, aqueous, aqueous chamber, vitreous, ciliary body, optic disc, papilla, and fovea.

Methods for inhibiting or preventing an ocular process associated with CTGF, the method comprising administering to a subject an agent that decreases expression or activity of CTGF or fragments thereof, wherein the ocular process is further associated with an ocular disorder, are specifically contemplated. Application of the present methods to any CTGF-associated ocular disorder is specifically contemplated. In particular embodiments, the ocular disorder is selected from the group consisting of glaucoma, cataract, choroidal neovascularization, retinal detachment, proliferative vitreoretinopathy, macular degeneration, diabetic retinopathy, corneal scarring, and corneal haze. In a preferred embodiment, the ocular process is further associated with ocular fibrosis.

A method for inhibiting or preventing ocular extracellular matrix production or deposition, the method comprising administering to a subject an agent that decreases expression or activity of CTGF or fragments thereof, is specifically provided herein. A method for inhibiting or preventing ocular scarring, the method comprising administering to a subject an agent that decreases expression or activity of CTGF or fragments thereof, is specifically provided. In one embodiment, the present invention provides a method for inhibiting or preventing ocular neovascularization, the method comprising administering to a subject an agent that decreases expression or activity of CTGF or fragments thereof In further embodiments, the neovascularization is retinal neovascularization, choroidal neovascularization, neovascularization of the iris, or trabecular neovascularization. A method for inhibiting or preventing ocular inflammation, the method comprising administering to a subject an agent that decreases expression or activity of CTGF or fragments thereof, is also provided.

The present invention further contemplates a method for inhibiting or preventing ocular cell proliferation, the method comprising administering to a subject an agent that decreases expression or activity of CTGF or fragments thereof. In various embodiments, the ocular cell proliferation is selected from the group consisting of epithelial cell proliferation, endothelial cell proliferation, retinal pigment epithelial cell proliferation, and choroidal endothelial cell proliferation.

In one aspect, the present invention provides a method for inhibiting or preventing ocular cell migration, the method comprising administering to a subject an agent that decreases expression or activity of CTGF or fragments thereof. In particular aspects, the ocular cell migration is selected from the group consisting of epithelial cell migration, retinal pigment epithelial cell migration, endothelial cell migration, and choroidal endothelial cell migration.

The present invention further provides a method for inhibiting or preventing an ocular process associated with CTGF in a subject, the method comprising administering an agent that decreases expression or activity of CTGF or fragments thereof, wherein the ocular process is further associated with surgery. In various aspects, the surgery is selected from the group consisting of refraction correction surgery, radial keratotomy, LASIK, retinal detachment surgery, corneal transplantation, glaucoma filtration surgery, cataract extraction surgery, lens replacement surgery, vitrectomy, subretinal surgery, and retinal translocation surgery.

Methods for Treating and Preventing Ocular Processes and Ocular Disorders

Methods for treating or preventing ocular disorders are specifically provided by the present invention. In one embodiment, the present invention provides a method for treating or preventing an ocular disorder, the method comprising administering to a subject an agent that decreases expression or activity of CTGF or fragments thereof. In another embodiment, a method for treating or preventing ocular fibrosis, the method comprising administering to a subject an agent that decreases expression or activity of CTGF or fragments thereof, is specifically contemplated.

In one aspect, the present invention provides a method for treating or preventing an ocular disorder associated with neovascularization, the method comprising administering to a subject an agent that decreases expression or activity of CTGF or fragments thereof. In various aspects, the neovascularization is retinal neovascularization, choroidal neovascularization, neovascularization of the iris, or trabecular neovascularization. In a particular aspect, a method for treating or preventing choroidal neovascularization; associated with macular degeneration is specifically contemplated.

A method for treating or preventing an ocular disorder associated with extracellular matrix production or deposition, the method comprising administering to a subject an agent that decreases expression or activity of CTGF or fragments thereof, is further provided, as is a method for treating or preventing an ocular disorder associated with inflammation, the method comprising administering to a subject an agent that decreases expression or activity of CTGF or fragments thereof.

In one embodiment, the present invention contemplates a method for treating or preventing an ocular disorder associated with cell proliferation, the method comprising administering to a subject an agent that decreases expression or activity of CTGF or fragments thereof. In various embodiments, the cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Müller cell, a perivascular astrocyte, a rod, a cone, a ganglion cell, and a bipolar cell.

In another embodiment, a method for treating or preventing an ocular disorder associated with cell migration, the method comprising administering to a subject an agent that decreases expression or activity of CTGF or fragments thereof, is provided. In certain embodiments, the cell is selected from the group consisting of an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Miller cell, a perivascular astrocyte, a rod, a cone, a ganglion cell, and a bipolar cell. In certain aspects, the present invention provides methods for treating ocular disorders associated with altered ocular cell migration, proliferation, chemotaxis, or attachment.

A method for treating or preventing a disorder associated with formation or alteration of an ocular membrane, the method comprising administering to a subject an agent that decreases expression or activity of CTGF or fragments thereof, is specifically contemplated. In various aspects, the ocular membrane is selected from the group consisting of an epiretinal membrane, a subretinal membrane, a vitreous membrane, a cellular membrane, a choroidal neovascularization membrane, and a fibrotic membrane. In a particular embodiment, the present invention provides a method for maintaining or improving vision in a subject, the method comprising administering to the subject an effective amount of an agent that decreases expression or activity of CTGF or fragments thereof.

In certain embodiments, the methods for treating or preventing an ocular disorder are administered prior to, at the time of, or in close association with surgery, in order to treat or to prevent the development of ocular disorders that can develop in association with a surgical event. In various aspects, the surgery is selected from the group consisting of refraction correction surgery, radial keratotomy, LASIK, retinal detachment surgery, corneal transplantation, glaucoma filtration surgery, cataract extraction surgery, lens replacement surgery, vitrectomy, subretinal surgery, and retinal translocation surgery.

The present invention provides a method for treating or preventing macular degeneration. Any of the methods described in the present invention can be applied to treat or prevent any form of macular degeneration, including age-related macular degeneration, dry form of macular degeneration, also known as non-exudative, involutional, atrophic macular degeneration, and geographic atrophy when severe, and wet form of macular degeneration, also know as exudative, neovascular, or disciform macular degeneration. In a preferred embodiment, the present invention provides a method for treating or preventing choroidal neovascularization associated with macular degeneration. In another embodiment, the present invention provides a method for treating or preventing diabetic retinopathy, including proliferative diabetic retinopathy and non-proliferative diabetic retinopathy. In a preferred embodiment, the present invention provides a method for treating or preventing proliferative vitreoretinopathy. In another aspect, the present invention provides a method of treating or preventing retinal detachment. In yet another aspect, the present invention provides a method for treating or preventing glaucoma. In still another aspect, the present invention provides a method of treating or preventing cataracts. In one embodiment, the present invention provides a method of treating or preventing corneal scarring. In another embodiment, the present invention provides a method of treating or preventing corneal haze.

The present invention provides a method for maintaining or improving vision in a subject, the method comprising administering to the subject an effective amount of an agent that decreases expression or activity of CTGF or fragments thereof.

Methods for Diagnosis

Methods for diagnosing an ocular disorder are specifically contemplated. In one embodiment, the present invention provides a method for diagnosing an ocular disorder associated with CTGF or fragments thereof, the method comprising obtaining a sample from a subject and measuring the level of CTGF or fragments of CTGF in the sample, and comparing the level of CTGF or fragments thereof in the sample to a standard level, wherein an increased amount of CTGF or fragments thereof in the sample is indicative of the presence of an ocular disorder. In another embodiment, the present invention provides a method for identifying a predisposition to develop an ocular disorder associated with CTGF, the method comprising obtaining a sample from a subject and measuring the level of CTGF or fragments thereof in the sample, and comparing the level of CTGF or fragments thereof in the sample to a standard level, wherein an increased amount of CTGF or fragments thereof in the sample is indicative of a predisposition to develop an ocular disorder.

The present invention also provides diagnostic kits for use in diagnosing an ocular disorder associated with CTGF. Diagnostic kits of the present invention comprise a means for detecting the level of CTGF in a sample and a means for measuring the level of CTGF in the sample. In one aspect, the diagnostic kit is useful for diagnosing an ocular disorder associated with CTGF. In another aspect, the diagnostic kit is useful for identifying a predisposition to develop an ocular disorder associated with CTGF. In a preferred embodiment, a diagnostic kit of the present invention is useful for diagnosing ocular fibrosis. In another preferred embodiment, a diagnostic kit of the present invention is useful for identifying a predisposition to develop ocular fibrosis.

Another aspect of the present invention provides methods for diagnosing ocular disorders, such as PVR, diabetic retinopathy, macular degeneration, or any ocular condition associated with expression or activity of CTGF or fragments thereof.

In a preferred method, diagnosis of an ocular disorder is accomplished by quantitating the level of CTGF or fragments of CTGF in a sample obtained from a subject. In one embodiment, the subject is a human subject. In another embodiment, the method includes determining the level of CTGF or fragments of CTGF in a tissue biopsy sample and comparing this level to the levels of CTGF or fragments of CTGF in a tissue biopsy from a normal tissue or sample, i.e., a sample from a subject without an ocular disorder associated with CTGF. An elevated level of CTGF or fragments of CTGF in the first sample is indicative of the pathological condition in question. (See, e.g., U.S. patent application Ser. No. 10/245,977, filed Sep. 18, 2002, incorporated herein by reference in its entirety.)

More generally, detection of CTGF or fragments of CTGF may be obtained through immunoassay methods, for example, using ELISAs, RIAs, or any other assays which utilize an antibody to detect the presence of a protein marker. The ELISA and RIA methods are preferred and may be used, for example, with the polyclonal or monoclonal antibodies of the present invention to detect levels of CTGF or fragments of CTGF. Levels of CTGF or fragments of CTGF in a first standard sample are determined, for example, through immunoassay, and are compared with the CTGF or fragments of CTGF levels in a second sample, the second sample being obtained from a patient known to have a CTGF-associated ocular disorder or from a patient known not to have any CTGF-associated ocular disorder, to determine the presence or progression of such a disorder.

More generally, antibodies specific for a target polypeptide, such as antibodies specific for CTGF or fragments thereof, are useful in the present invention for diagnosis of CTGF-associated ocular disorders. The present diagnostic assays include methods utilizing the antibody and a label to detect CTGF or fragments thereof, in a sample from a patient suspected of having a CTGF-associated ocular disorder. The sample could comprise, for example, body fluids (e.g., vitreous humor), cells, tissues, or extracts of such tissues, including, for example, cells micro-dissected from biopsy material. Protocols employed to screen for and identify antibodies having the desired specificity can also be used for the detection of CTGF or fragments thereof in the sample.

Preferably, in the diagnostic methods of the present invention, normal or standard values for CTGF or CTGF fragment expression or activity are established in order to provide a basis for the diagnosis of the existence of a CTGF-associated ocular disorder or a predisposition to a CTGF-associated ocular disorder. In one method of the present invention, this is accomplished by combining body fluids (e.g., vitreous humor), tissue biopsies, or cell extracts taken from normal subjects with antibody to CTGF or fragments of CTGF under conditions suitable for complex formation. Such conditions are well known in the art. The amount of standard complex formation may be quantified by comparing levels of antibody-target complex in the normal sample with a dilution series of positive controls, in which a known amount of antibody is combined with known concentrations of purified CTGF or fragments thereof. Standard values obtained from normal samples may be compared, for example, in a specific embodiment, with values obtained from samples from subjects suspected of having a CTGF-associated ocular disorder, or having a predisposition to a CTGF-associated ocular disorder. Deviation between standard and subject values establishes the presence of or predisposition to the disease state. The diagnostic methods of the present invention may also be directed to the detection of a predisposition or susceptibility to an ocular disorder, such as, for example, PVR, diabetic retinopathy, or macular degeneration.

The present invention provides kits for detecting CTGF or CTGF fragments in samples, in particular, in fluid samples or tissue biopsy samples. In a particular embodiment, this kit comprises a monoclonal and/or polyclonal antibody specific for CTGF or fragments of CTGF, bound to a support and a second monoclonal and/or polyclonal antibody specific for a different CTGF or CTGF fragment epitope and enzyme-labeled. The kit further comprises reagents for detecting the enzyme-labeled monoclonal and/or polyclonal antibody. The reagent kit employs immunological methods in measuring CTGF or fragments thereof in the sample, thus allowing for the detection and diagnosis of ocular disorders, in particular CTGF-associated ocular disorders. In another embodiment, the kit comprises a radio-labeled or fluorescein labeled antibody in place of the enzyme-labeled antibody.

In one embodiment, the diagnostic kit of the present invention comprises elements useful in the detection of CTGF or fragments of CTGF in tissue samples, using immuno-histochemical techniques. The kit could be used in conjunction with, for example, a software program which allows for quantitative measurement of the levels of CTGF or fragments of CTGF in the tissue sample by image analysis or other comparative techniques. Another embodiment provides a diagnostic kit for detecting and measuring levels of CTGF mRNA in tissue samples. In one embodiment, the kit comprises reagents used to reverse transcribe CTGF mRNA to DNA. The kit can further comprise reagents necessary to amplify CTGF-specific DNA, including primers complementary to polynucleotides encoding CTGF, or fragments thereof. The kit can also include a competitive mimic or mutant cDNA for use in quantifying the level CTGF mRNA present in the sample.

In a preferred embodiment, the diagnostic kit of the present invention is packaged and labeled, for example, in box or container which includes the necessary elements of the kit, and includes directions and instructions on the use of the diagnostic kit.

Methods for Screening

Methods for identifying an agent useful for inhibiting or preventing an ocular process are also provided by the present invention.

In one embodiment, the present invention provides a method for identifying an agent for use in inhibiting or preventing an ocular process associated with CTGF, the method comprising contacting a candidate agent with CTGF or a fragment thereof, detecting the level of CTGF expression or activity in the sample, and comparing the level of CTGF expression or activity in the sample to a standard level of CTGF expression or activity, wherein decreased expression and activity of CTGF or fragments thereof in the sample is indicative of an agent that inhibits or prevents an ocular process associated with CTGF.

In another embodiment, the present invention provides a method for identifying an agent for use in treating or preventing an ocular disorder associated with. CTGF, the method comprising contacting a candidate agent with CTGF or a fragment thereof detecting the level of CTGF expression or activity in the sample, and comparing the level of CTGF expression or activity in the sample to a standard level of CTGF expression or activity, wherein decreased expression and activity of CTGF or fragments thereof in the sample is indicative of an agent that treats or prevents an ocular disorder associated with CTGF or fragments thereof.

The present invention provides methods for screening or identifying agents that can be used in methods for treating or preventing ocular processes and associated ocular disorders. Compounds identified using the present screening methods can be administered to a subject to produce the desired effect, such as decreasing expression or activity of CTGF or CTGF fragments. Additionally, the present invention provides methods for the identifying agents that decrease expression or activity of CTGF or CTGF fragments in specific ocular cells, such as RPE cells, CECs, etc.

The present invention contemplates methods for screening or otherwise identifying useful agents, including agonists or antagonists, which can specifically recognize CTGF or fragments of CTGF. Agents that bind to CTGF or fragments of CTGF may inhibit the activities of these polypeptides. The agents can include, for example, antibodies and fragments thereof, small molecules, polypeptides (synthetic, natural, or enzymatically- or recombinantly-produced), and aptamers. The present invention additionally contemplates methods useful to identify agents for use in treating or preventing ocular disorders by decreasing the activity or expression of CTGF or fragments thereof.

In one aspect, screening assays of the present invention include contacting CTGF or fragments of CTGF with the candidate agent, detecting a level of CTGF activity or expression, and comparing that level of activity or expression to a standard level obtained by methods known in the art. These methods could involve, for example, CTGF or fragments of CTGF affixed to solid supports, cell-free preparations, or natural or synthetic product mixtures. Assays, such as ELISAs, can be designed in which antibodies, monoclonal or polyclonal, bind directly or indirectly to CTGF or to fragments of CTGF or compete with CTGF or with fragments of CTGF for binding.

Other techniques for screening which provide for high throughput screening of agents having suitable binding affinity to CTGF or to fragments of CTGF, or to another target polypeptide useful in modulating, regulating, or inhibiting the expression or activity of CTGF or fragments of CTGF, are known in the art. For example, microarrays carrying test compounds can be prepared, used, and analyzed using methods available in the art. (See, e.g., International Publication No. WO95/35505; International Publication No. WO95/251116; U.S. Pat. No. 5,474,796; U.S. Pat. No. 5,605,662.)

Identifying small agents that decrease CTGF activity or expression can also be conducted by various other screening techniques, which can serve to identify antibodies and other agents that interact with a CTGF or fragments of CTGF and can be used as drugs and therapeutics in the present methods. (See, e.g., Enna et al. (1998) Current Protocols in Pharmacology, John Wiley and Sons.) Assays will typically provide for detectable signals associated with the binding of the compound to a protein or cellular target. Binding can be detected by, for example, fluorophores, enzyme conjugates, and other detectable labels well-known in the art. (Id.) The results may be qualitative or quantitative.

Pharmaceutical Formulations and Routes of Administration

The present invention contemplates methods of treatment in which compounds or agents that modulate the activity or expression of CTGF are administered, for example, in vivo, to affect the activity of CTGF. These agents can be delivered directly or in pharmaceutical compositions along with suitable carriers or excipients, as well known in the art. Present methods of treatment can comprise administration of an effective amount of an agent that inhibits the activity or expression of CTGF or fragments of CTGF to a subject in need of treatment. In a preferred embodiment, the subject is a mammalian subject, and in a most preferred embodiment, the subject is a human subject. Various formulations and drug delivery systems are available in the art. (See, e.g., Remington's Pharmaceutical Sciences, supra.) In one aspect, the agent is selected from the group consisting of an antibody, an antisense oligonucleotide, and a small molecule. In another aspect, the agent is delivered to the eye. Methods in which the agent is delivered to the ocular surface are specifically contemplated. In a preferred aspect, the agent is delivered in an eye drop formulation. In another aspect, the agent is delivered as an ointment.

Suitable routes of administration may, for example, include oral, rectal, transmucosal, nasal, or intestinal administration and parenteral delivery, including intramuscular, subcutaneous, intramedullary injections, as well as intrathecal, direct intraventricular, intravenous, intraperitoneal, intranasal, or intraocular injections. The agent may be administered in a local or a systemic manner. For example, a suitable agent can be delivered via injection or in a targeted drug delivery system, such as depot, catheter, or sustained release formulation. Local ocular administration can include, for example, intraocular, intravitreal, subretinal, subscleral, subconjunttival, subtenon, or intrascleral, injection or application. Suitable agents can also be applied directly to the cornea or sclera.

For any composition or agent used in the present methods of treatment, a therapeutically effective dose can be estimated initially using a variety of techniques well known in the art. For example, in a cell culture assay, a dose can be formulated in animal models to achieve a circulating concentration range that includes the IC₅₀ as determined in cell culture. Where inhibition of CTGF expression or activity is desired, for example, the concentration of the test agent that achieves a half-maximal inhibition of CTGF activity can be determined. Dosage ranges appropriate for human subjects can be determined, for example, using data obtained from cell culture assays and other animal studies. A therapeutically effective dose of an agent refers to that amount of the agent that results in amelioration of symptoms or a prolongation of survival in a subject.

Antibodies

Antibodies directed to CTGF or fragments of CTGF may be generated using methods well known in the art. Such antibodies may include, but are not limited to, polyclonal, monoclonal, chimeric, and single chain antibodies, as well as Fab fragments, including F(ab′)₂ and Fv fragments. Fragments can be produced, for example, by a Fab expression library. Neutralizing antibodies, i.e., those which inhibit CTGF activity, are especially preferred for therapeutic use.

Methods for the production of antibodies are well known in the art. For example, various hosts, including goats, rabbits, rats, mice, chickens, turkeys, humans, and others, may be immunized by injection with the target polypeptide or any immunogenic fragment or peptide thereof. Techniques for in vivo and in vitro production of either monoclonal or polyclonal antibodies are well known in the art. (See, e.g., Pound (1998) Immunochemical Protocols, Humana Press, Totowa N.J.; Harlow and Lane (1988) Antibodies: A Laboratory Manual, Cold Spring Harbor Laboratory, New York; Goding (1986) Monoclonal Antibodies: Principles and Practice, 2^{nd} Edition, Academic Press; Schook (1987) Monoclonal Antibody Production Techniques and Applications, Marcel Dekker, Inc.)

Antibodies as described above could also be used to identify CTGF, or fragments or subunits thereof, in a sample, e.g., from blood, biopsied tissue, vitreous, tears, etc. The amount of CTGF or fragments of CTGF present could be determined, for example, by quantitative image analysis, ELISA, or imuunohistochemical techniques. CTGF mRNA levels could also be determined, e.g., by reverse transcriptase polymerase chain reaction (RT-PCR), using portions of the biopsied tissue, or by other methods well known in the art. In particular, in this method, mRNA from a tissue sample, in total, or that specific for CTGF or fragments or subunits thereof, could be transcribed to DNA and then amplified through PCR using specific primer sequences. Quantitation of mRNA corresponding to CTGF could be determined, for example, by a competition reaction using equal volumes of the patient sample run against a series of decreasing known concentrations, e.g., of a mimic or mutant cDNA fragment.

The present invention contemplates the use of antibodies specifically reactive with CTGF or fragments or subunits thereof that neutralize the biological activity of CTGF or fragments of CTGF. The antibody administered in the method can be an intact antibody or antigen binding fragments thereof, such as Fab, F(ab′)₂, and Fv fragments, which are capable of binding the epitopic determinant. The antibodies used in the method can be polyclonal or, more preferably, monoclonal antibodies. Monoclonal antibodies with different epitopic specificities are made from antigen-containing fragments of the protein by methods well known in the art. (See Ausubel et al., supra.)

In the present invention, therapeutic applications include those using “human” or “humanized” antibodies directed to CTGF or fragments thereof. Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for CTGF is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

Antisense

The present invention provides for a therapeutic approach which uses antisense technology to inhibit expression and activity of CTGF or fragments thereof. Specifically, a therapeutic approach which directly interrupts the transcription of DNA encoding CTGF or translation of CTGF mRNA could be used to alter the expression and activity of CTGF and of fragments of CTGF.

Antisense technology relies on the modulation of expression of a target protein through the specific binding of an antisense sequence to a target sequence encoding the target protein or directing its expression. (See, e.g., Agrawal, S., ed. (1996) Antisense Therapeutics, Humana Press Inc., Totawa N.J.; Alama et al. (1997) Pharmacol Res 36(3):171-178; Crooke (1997) Adv Pharmacol 40:1-49; Lavrosky et al. (1997) Biochem Mol Med 62(1):11-22.)

Delivery of antisense sequences can be accomplished in a variety of ways, such as, for example, through intracellular delivery using an expression vector or direct administration of antisense oligonucleotides.

Delivery of antisense therapies and the like can be achieved intracellularly through using a recombinant expression vector such as a chimeric virus or a colloidal dispersion system which, upon transcription, produces a sequence complementary to at least a portion of the cellular sequence encoding the target protein. (See, e.g., Slater et al. (1998) J Allergy Cli Immunol 102(3):469-475.) Delivery of antisense sequences can also be achieved through various viral vectors, including retrovirus and adeno-associated virus vectors. (See, e.g., Miller (1990) Blood 76:271; Uckert and Walther (1994) Pharacol Ther 63(3):323-347.) Vectors which can be utilized for antisense gene therapy as taught herein include, but are not limited to, adenoviruses, herpes viruses, vaccinia, or, preferably, RNA viruses such as retroviruses.

Other gene delivery mechanisms that can be used for delivery of antisense sequences to target cells include colloidal dispersion and liposome-derived systems, artificial viral envelopes, and other systems available to one of skill in the art. (See, e.g., Rossi (1995) Br Med Bull 51(1):217-225; Morris et al. (1997) Nucl Acids Res 25(14):2730-2736; Boado et al. (1998) J Pharm Sci 87(11):1308-1315.) For example, delivery systems can make use of macromolecule complexes, nanocapsules, microspheres, beads, and lipid-based systems including oil-in-water emulsions, micelles, mixed micelles, and liposomes.

The following examples explain in the invention in more detail. The following preparations and examples are given to enable those of skill in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those of skill in the art from the foregoing description and accompanying drawings. Such modifications are intended to fall within the scope of the appended claims.

### EXAMPLES

### Example 1

### RPE Cells and CECs Express CTGF mRNA

Human RPE cells were derived as follows. Human fetal eyes (18 to 26 weeks of gestation) were obtained from the Anatomic Gift Foundation (Woodbine, Ga.). RPE cells were isolated from human fetal eyes using methods previously described. (Jin et al. (2000) IOVS 41:4324-4332.) RPE cells at passage two to four were used in all studies. RPE cells were cultured in Dulbecco's Eagles Medium (Fisher Scientific, Pittsburgh, Pa.) supplemented with 10% fetal bovine serum (Gibco/BRL, Gathersbery, Md.), 2 mM glutamine, 100 μg/ml streptomycin, and 100 μg/ml penicillin (Sigma Chemical Co., St. Louis, Mo.). The purity of cultured RPE cells was evaluated by immuno-cytochemical staining using an antibody against cytokeratin (Catalog No. M0821, Dako, Carpinteria, Calif.), endothelial cell antigen von Willebrand factor (Catalog No. M0616, Dako), and macrophage antigen CD11c (Catalog No. M0732, Dako). Cultured RPE cells used in these studies were typically 99% pure based on immuno-cytochemical staining performed using these cell-type specific markers.

CECs were isolated from bovine eyes using previously described methods. (Hoffmann et al. (1998) Graefes Arch Clin Exp Ophthalmol 236:779-784.) Briefly, magnetic beads (Dynabeads) coated with Lycopersicon esculentum (Sigma Chemical Co.), a specific endothelial cell marker, were used to isolate CECs from bovine eyes. CECs isolated from bovine eyes were confirmed to be vascular endothelial cells by positive immunostaining for von Willebrand factor, as well as by the ability of the isolated CECs to bind dil-acetylated low-density lipoprotein. (Hoffman et al. supra.)

Expression of CTGF mRNA was examined in cultured human RPE cells and in cultured bovine CECs using reverse transcriptase polymerase chain reaction (RT-PCR). Poly(A)⁺ RNA was isolated from RPE cells and CECs using a FastTrack Kit (Invitrogen, San Diego, Calif.), according to methods described by the manufacturer. First-strand cDNA was synthesized at 42° C. using M-MLV reverse transcriptase (Gibco/BRL) with an oligo-d(T) primer (Catalog No. C110A, Promega, Madison, Wis.). PCR was performed using oligonucleotide sequence primers specific for human CTGF or bovine CTGF. The primer sequences used for PCR for amplification of human CTGF sequence from RPE cells were 5′-gcatccgtactcccaaaatc-3′ (SEQ ID NO:1) and 5′-cttcttcatgacctcgccgt-3′ (SEQ ID NO:2). The primer sequences used for PCR amplification of bovine CTGF sequence from CECs were 5′-gatcataggactgtattagtgc-3′ (SEQ ID NO:3) and 5′-ctgacttagagacgaacttg-3′ (SEQ ID NO:4). PCR was performed using a Perkin-Elmer/Centur DNA thermal cycler, using the following conditions: denaturation at 94° C. for 1 minute; annealing at 60° C. for 1 minute; and extension at 72° C. for 45 seconds. Following 30 cycles of PCR, the amplified products were resolved on 1.2% agarose gels and viewed following staining of the gel with ethidium bromide. As expected, the use of these primers resulted in CTGF-specific DNA amplification products of 210 base pairs (human CTGF) and 220 base pairs (bovine CTGF) in size.

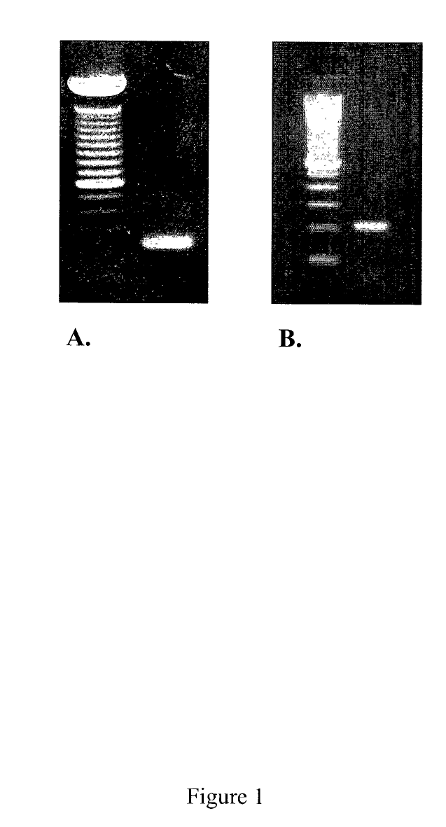

CTGF mRNA was detected in cultured RPE cells and in CECs. As shown in FIG. 1A and FIG. 1B (right lanes), both RPE cells and CECs were positive for CTGF mRNA expression, as evidenced by the presence of the expected 210 base pair and 220 base pair CTGF RT-PCR amplification products. The molecular weight markers shown in FIG. 1A and FIG. 1B (left lanes) is a 100 base pair DNA ladder (Gibco/BRL). CTGF mRNA was not observed in adult RPE cells (data not shown). These results demonstrated that RPE cells and CECs, which are involved in the pathogenesis of many ocular disorders, including the development of PVR and CNV, express CTGF mRNA. Therefore, various ocular cells are capable of expressing CTGF mRNA.

### Example 2

### RPE Cells and CECs Express CTGF Polypeptide

The expression of CTGF polypeptide by RPE cells and CECs was investigated. RPE cells and CECs were isolated as described above in Example 1 and cultured in 6-well tissue culture plates. Cultured RPE cells were starved for 24 hours in serum-free DMEM. Cultured CECs were starved for 24 hours in serum-free Essential Basal Medium (EBM) (BioWhittaker, Walkersville, Md.), containing 0.1% bovine serum albumin, for 24 hours. Serum-free RPE cell media was replaced with fresh serum-free media containing transforming growth factor beta2 (TGFβ2) (1 to 30 ng/ml) or vascular endothelial growth factor (VEGF) (10 to 50 ng/ml). Serum-free CEC media was replaced with fresh media containing 1% fetal bovine serum containing recombinant VEGF (10 to 50 ng/ml) or TGFβ2 (1 to 30 ng/ml). Both cell types were incubated for an additional 48 hours in the presence of growth factor. Cell lysates were collected and electrophoresed on TRIS-HCl 10% polyacrylamide gels (Ready Gel, Bio-Rad Laboratories, Hercules, Calif.) and the proteins were transferred from the gels to PVDF blotting membranes (Millipore, Bedford, Mass.). The presence of CTGF in cell lysates was examined by western blot analysis. The membranes were probed for CTGF using a polyclonal rabbit anti-CTGF antibody, prepared using standard methods known in the art, followed by HRP-conjugated goat anti-rabbit secondary antibody (Vector Laboratories, Burlingame, Calif.), using standard methods. Image development was performed with the addition of ECL chemiluminescent detection solution (Amersham Pharmacia Biotech, Cleveland, Ohio) and exposure to Hyperfilm (Amersham Pharmacia Biotech).

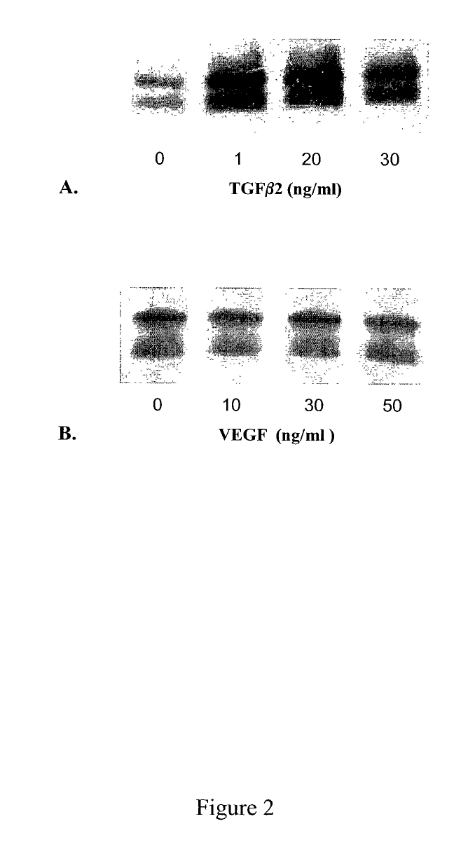

Cultured RPE cells expressed CTGF polypeptide. As the western blots in FIG. 2A and FIG. 2B show, in the absence of TGFβ2 or VEGF addition, detectable levels of CTGF protein were observed in untreated RPE cell lysates. Stimulation of RPE cells with various concentrations of TGFβ2 (1 to 30 ng/ml) for 48 hours in serum-free media resulted in increased CTGF protein expression compared to non-treated control cells. (FIG. 2A.) The stimulation of CTGF expression in RPE cells by TGFβ2 was dose-dependent, as shown in FIG. 2A. Increased expression of CTGF (above that of control) was observed in RPE cells treated with TGFβ2 concentrations as low as 1 ng/ml. These results showed that RPE cells synthesized CTGF polypeptides, and that CTGF expression by RPE cells was up-regulated by TGFβ2 in a dose-dependant manner. No increase in CTGF expression was detected in RPE cells treated with VEGF was detected. (FIG. 2B.)

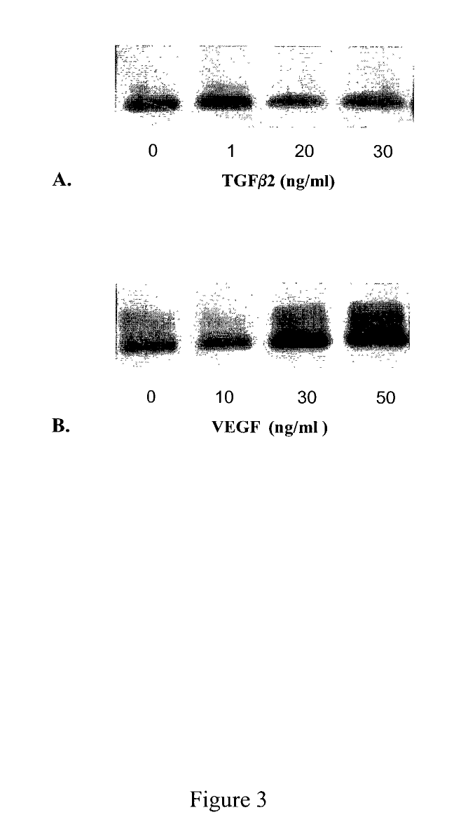

Cultured CECs were also found to express CTGF protein. The western blots in FIG. 3A and FIG. 3B show that detectable levels of CTGF protein were observed in lysates from untreated CECs. Stimulation of CECs with various concentrations of VEGF (10 to 50 ng/ml) for 48 hours in low-serum media resulted in increased CTGF protein expression compared to non-treated control cells. Stimulation of CTGF expression in CECs by VEGF was dose-dependent, as shown in FIG. 3B. No increase in CTGF expression was detected in CECs treated with TGFβ2. (FIG. 3A.) Taken together, these results demonstrated that RPE cells and CECs, which have important roles in the pathogenesis of many ocular disorders, such as PVR and CNV, express CTGF polypeptides and that CTGF expression in these cells is differentially regulated by TGFβ2 and VEGF.

In addition to showing that ocular cells expressed CTGF protein, these results also demonstrated that CTGF expression in ocular cells is regulated by various growth factors and cytokines which are located within the eye. Additionally, these results showed that differential regulation of CTGF expression occurred in various ocular cells. Specifically, TGFβ2 stimulated increased expression of CTGF in RPE cells. Within the eye, vitreous contains high levels of TGFβ. (Limb et al. (1991) Eye 5: 686-693.) Various ocular disorders are associated with RPE cell exposure to vitreous. Therefore, the initiation, progression, or maintenance of various ocular disorders may result from TGFβ stimulation of CTGF expression in ocular cells, such as RPE. Increased expression of VEGF in ocular neovascularization has been reported. (Lopez et al. (1996) Invest Ophthalmol Vis Sci 37:855-868; Simo et al. (2002) Am J Ophthalmol 134: 376-382.) Therefore, various ocular disorders associated with increased expression of VEGF show increased CTGF when choroidal or retinal endothelial are exposed to VEGF.

CTGF levels in the supernatants of cultured RPE cells were determined by ELISA analysis. Cultured human RPE cells were treated with or without TGFβ2 (20 ng/ml) for 48 hours, as described above. Cell supernatants were harvested and cleared by centrifugation at 5,000 rpm for 5 minutes. Samples were stored at −70° C. until ELISA assays were performed. ELISA data indicated that non-treated control RPE cells secreted CTGF (9 ng/ml). RPE cells treated with TGFβ2 secreted increased levels of CTGF (47 ng/ml). Taken together, these results indicated that ocular cells synthesize and secrete CTGF. Additionally, these results showed that CTGF synthesized by ocular cells can be measured qualitatively and quantitatively.

### Example 3

### Production of Recombinant Human CTGF

Recombinant human CTGF (rhCTGF) was prepared as follows. A full-length human CTGF cDNA (DB60R32) was obtained from Dr. Gary Grotendorst (University of Miami Medical School). (Bradham et al. (1991) J Cell Biol 114:1285-1294.) A CTGF cDNA comprising only the open reading frame was generated by the polymerase chain reaction using DB60R32 DNA as template and the following primers (5′-gctccgcccgcagtgggatccatgaccgccgcc-3′ (SEQ ID NO:5) and 5′-ggatccggatcctcatgccatgtctccgta-3′) (SEQ ID NO:6), which added BamHI restriction enzyme sites (underlined) to either end of the amplified product. The resulting amplified DNA fragment was digested with BamHI, purified on an agarose gel, and subcloned directly into the BamHI site of the baculovirus (donor) expression plasmid pFastBac1 (Invitrogen, Carlsbad, Calif.). The pFastBac1/CTGF cDNA vector construct was transposed into bacmid DNA and recombinant baculovirus was generated by following the manufacturer's protocol outlined in the BAC-TO-BAC Baculovirus Expression System manual (Invitrogen). Expansion of recombinant baculovirus titers in Sf9 insect cells was performed using standard procedures known in the art. (Murphy and Piwnica-Worms, (1984) Current Protocols in Molecular Biology, Vol. 2 (Ausubel et al., Eds.) John Wiley & Sons, Inc.)

Expression and production of rhCTGF was performed as follows. HIGH FIVE insect cells, adapted to suspension growth, were grown in SF900II medium (Invitrogen) to a cell density of 1.0×10⁶ cells/ml. The cells were then infected with baculovirus containing CTGF at a multiplicity of infection of 10:1. Following infection, the cells were incubated at 27° C. for 40 hours. The cells were then pelleted by centrifugation, and the CTGF-containing conditioned medium was collected and filtered through a 0.22 μm filter. The conditioned medium was then directly applied to a 5 ml Hi-Trap heparin column (Amersham Biosciences Corp., Piscataway N.J.), which had been pre-equilibrated with 50 mM Tris, pH 7.2. The heparin column was then washed with 100 ml of 350 mM NaCl/50 mM Tris, pH 7.2, and bound rhCTGF was eluted with a linear gradient of 350 mM NaCl to 1200 mM NaCl over 15 column volumes. Fractions were analyzed for the presence of rhCTGF by SDS-PAGE.

Fractions containing rhCTGF were pooled and diluted 1:4 with 50 mM Tris, pH 7.5. The rhCTGF pool was loaded over a carboxy methyl (CM) ion exchange column (POROS CM column, PerSeptive Biosystems, Framingham, Mass.), which had been pre-equilibrated with 10 column volumes of 50 mM Tris, pH 7.5. The CM column was washed with 350 mM NaCl/50 mM Tris, pH 7.5, and bound rhCTGF was eluted with a linear gradient of 350 mM NaCl to 1200 mM NaCl over 15 column volumes. The fractions were analyzed for the presence of CTGF by SDS-PAGE. Fractions containing rhCTGF were pooled and used as rhCTGF material.

### Example 4

### Production of rhCTGF N-Terminal and C-Terminal Fragments

N-terminal fragments and C-terminal fragments of rhCTGF were prepared as follows. Recombinant human CTGF, prepared and purified as described above, was digested by treatment with chymotrypsin beads (Sigma Chemical Co., St. Louis Mo.), using 1.5 mg of rhCTGF per unit of chymotrypsin. Chymotrypsin treatment of rhCTGF was allowed to proceed at room temperature for 6 hours. The digested product and chymotrypsin beads were centrifuged, the chymotrypsin beads were discarded, and the supernatant, containing enyzmatically cleaved rhCTGF, was diluted 1:5 with 50 mM Tris, pH 7.5. The diluted supernatant was applied to a Hi-Trap heparin column. The flow-through was collected, and contained N-terminal fragments of rhCTGF. The heparin column was washed with 350 mM NaCl, and bound C-terminal fragments of rhCTGF and undigested rhCTGF were eluted with a linear gradient of 350 mM to 1200 mM NaCl, as described above. The fractions were analyzed for the presence of C-terminal fragments of rhCTGF by SDS-PAGE. Fractions containing C-terminal fragments of rhCTGF were pooled according to the observed purity of C-terminal rhCTGF.

The heparin column flow-through, which contained N-terminal fragments of rhCTGF, was adjusted to contain 0.5 M ammonium sulfate/50 mM Tris, pH 7.5. The sample containing N-terminal fragments of rhCTGF was then loaded onto a 15 ml phenyl sepharose HP column (Amersham Pharmacia Biotech), which had been pre-equilibrated with 0.5 M ammonium sulfate/50 mM Tris, pH 7.5. The phenyl sepharose HP column was then washed with 15 column volumes of 0.5 M ammonium sulfate/50 mM Tris, pH 7.5. Bound N-terminal fragments of rhCTGF were eluted with a linear gradient of 0.5 M to 0.0 M ammonium sulfate/50 mM Tris, pH 7.5, over approximately 15 column volumes. Fractions were analyzed for the presence of N-terminal fragments of rhCTGF by SDS-PAGE. Fractions containing N-terminal fragments of rhCTGF were pooled. This pool, containing N-terminal fragments of rhCTGF, was concentrated and the buffer exchanged with 50 mM Tris, 400 mM NaCl, pH 7.2, using Amicon ultrafiltration, YM10 membrane.

### Example 5

### CTGF Stimulates RPE Cell and CEC Attachment to Fibronectin

Extracellular matrix molecules play a crucial role in mediating various activities of ocular cells, including ocular cell differentiation, attachment, migration, and proliferation. To examine the role of CTGF in extracellular matrix production in ocular cells and ocular disorders, the effect of CTGF on RPE cell and CEC attachment to fibronectin was investigated by performing the following assay.

RPE cells or CECs (1×10⁵ cells/ml), isolated and cultured as described above in Example 1, were removed from tissue culture plates by trypsinization. RPE cells were resuspended in cell culture media containing 0.4% fetal bovine serum, allowed to attach to the plate, and incubated with various concentrations of rhCTGF (10 to 100 ng/ml) for 48 hours at 37° C. RPE cells were detached from the plate and 1×10⁴ cells (in 100 microlitres) were then added to 96-well tissue culture plates (Becton Dickinson Labware, Bedford, Mass.) previously coated with fibronectin (50 μg/ml). The cells were allowed to attach to the fibronectin-coated plates for 60 minutes at 37° C. The wells of the plates were then gently washed twice with phosphate-buffered saline (PBS) to remove non-attached cells. Fresh medium (150 μl) containing 20 μl of a 5 mg/ml solution of MTT (3-(4,5-dimethyl-2-thiazolyl)-2,5-diphenyl-2H tetrazolium bromide, Sigma Chemical Co.) was added to each well, and the plates, which contained attached cells, were incubated for 5 hours at 37° C. The supernatants were removed from the wells and formazan precipitates associated with attached cells were solubilized with 100 μl of 100% DMSO (Sigma Chemical Co.), followed by placement of the plates on a shaker for 10 minutes. Absorbance at 550 mm was determined using a Dynatech MR 600 microplate reader. This MTT assay provides a quantitative analysis of the measurement of the number of cells remaining attached to the fibronectin-coated tissue culture plates. This assay is performed following shaking of the plates, and therefore correlated with the strength of attachment of the cells to fibronectin. The number of attached cells was proportional to the absorbance measured in the MTT assay described.

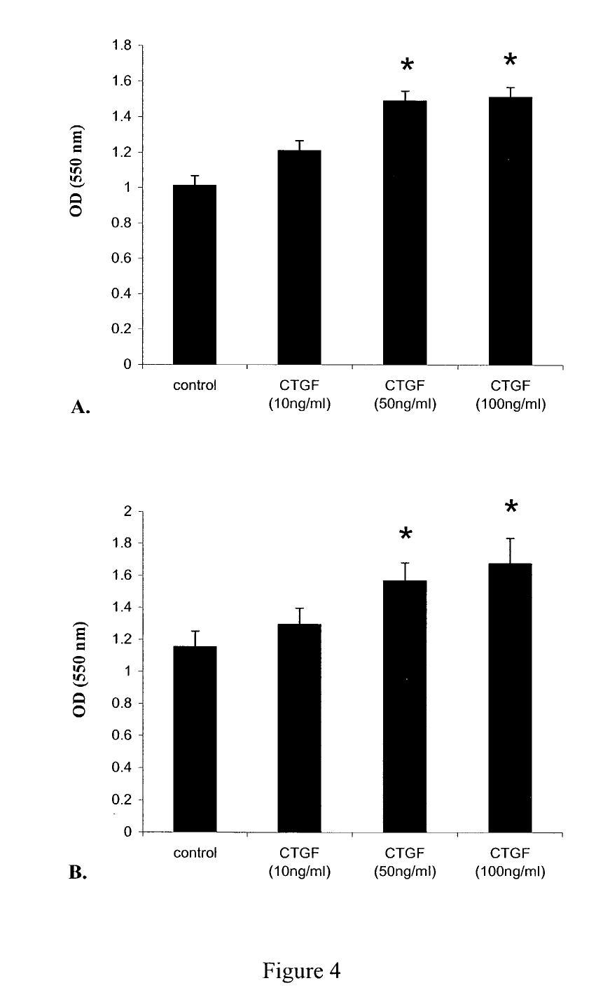

Both RPE cells (FIG. 4A) and CECs (FIG. 4B) showed increased attachment to fibronectin following treatment with rhCTGF compared to non-treated control cells. The effect of rhCTGF on RPE cell and CEC attachment to fibronectin was dose-dependant, as shown in FIG. 4A and FIG. 4B. In both RPE cells and CECs, a significant increase in cell attachment to fibronectin was observed in rhCTGF-treated cells, compared to control cells, at rhCTGF concentrations of 50 ng/ml or 100 ng/ml (indicated by asterisks (P<0.05) in FIG. 4A and FIG. 4B). These results indicated CTGF affects extracellular matrix production in ocular cells, at least in part by stimulating the production of fibronectin in RPE cells and CECs.

### Example 6

### CTGF Simulates Chemotaxis of RPE Cells and CECs

Ocular cell migration and chemotaxis are associated with many ocular disorders, including choroidal neovascularization and PVR. The effect of CTGF on chemotaxis of RPE cells and CECs was investigated. To examine chemotaxis, cell migration assays were performed using a modified Boyden chamber assay as previously described. (He et al. (1998) Biochem Biophys Res Commun 249:253-257.) RPE cells and CECs were isolated as described above in Example 1, and cultured on fibronectin-coated inserts (50 μg/ml) of 24-well Boyden chamber plates in media containing 0.4% fetal bovine serum. Various concentrations of rhCTGF (1 to 50 ng/ml) were added to the lower wells of the assay plates to examine the chemotactic activity of CTGF for RPE cells and CECs. The cells were then incubated for 5 hours, after which the chamber inserts were washed three times with PBS, fixed with ice-cold 100% methanol for 10 minutes, and counterstained with hematoxylin for 20 minutes. The number of cells that migrated was counted using phase-contrast microscopy (320×). The number of cells that migrated was determined in four randomly chosen fields per insert.

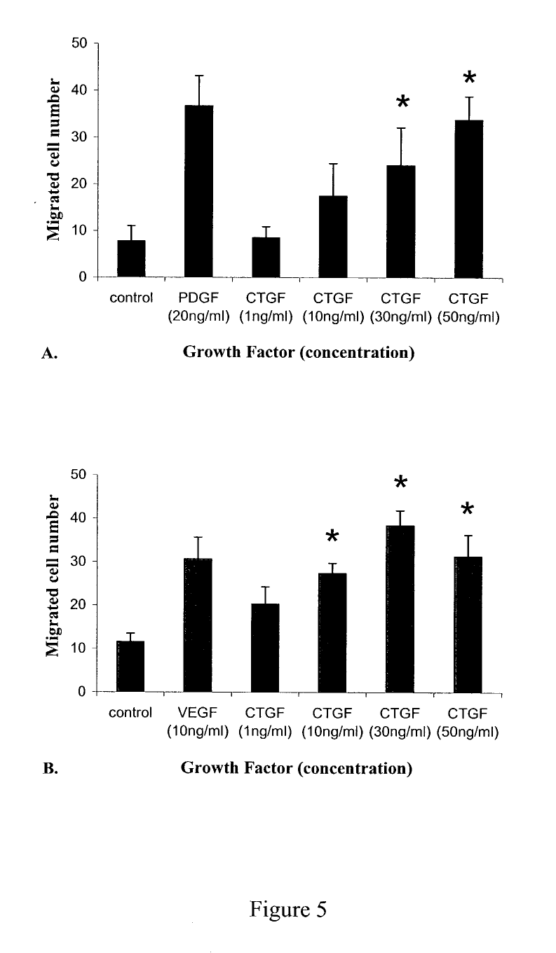

FIG. 5A and FIG. 5B show that rhCTGF stimulated migration and chemotaxis of RPE cells and CECs in a dose-dependant manner. As shown in FIG. 5A, a significant increase in RPE cell migration above control occurred following addition of either 30 ng/ml or 50 ng/ml rhCTGF (indicated by asterisks (P<0.01) in FIG. 5A). With the addition of 50 ng/ml rhCTGF, the extent of RPE cell migration was similar to that observed with platelet derived growth factor (PDGF) (20 ng/ml), a potent chemoattractant factor for RPE cells, which served as a positive chemoattractant factor control. Therefore, rhCTGF displayed potent chemotactant activity to RPE cells, and was equally effective at stimulating chemotaxis as PDGF, a known potent chemoattractant factor.

As shown in FIG. 5B, rhCTGF stimulated the migration and chemotaxis of CECs above that observed in non-treated control cells. Addition of rhCTGF at concentrations of 10 ng/ml, 30 ng/ml, and 50 ng/ml resulted in significant increases in CEC migration above non-treated controls (indicated by asterisks (P<0.05) in FIG. 5B). Addition of rhCTGF at concentrations of 30 ng/ml and 50 ng/ml were at least as effective at stimulating CEC migration as was VEGF (10 ng/ml), a known potent chemoattractant factor to CECs. Therefore, rhCTGF displayed potent chemotactant activity to CECs, and was equally as effective at stimulating chemotaxis in CECs as VEGF, a known potent chemotactic factor. Taken together, these results indicated CTGF was a potent chemotactic factor for various ocular cells.

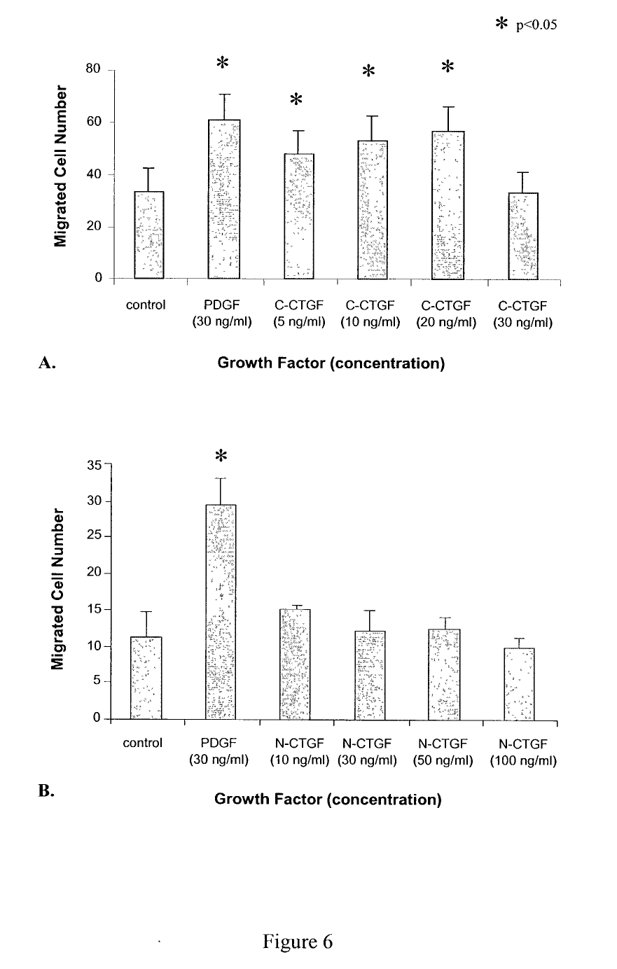

The effect of CTGF fragments on chemotaxis of RPE cells was also investigated. Various concentrations of recombinant human CTGF N-terminal fragment (10 to 100 ng/ml) and C-terminal fragment (5 to 30 ng/ml) were tested in the cell migration assay as described above. As shown in FIG. 6A, rhCTGF C-terminal fragments stimulated migration and chemotaxis of RPE cells in a dose-dependent manner. A maximal chemotactic effect of CTGF fragments in RPE cells was observed upon addition of 20 ng/ml rhCTGF C-terminal fragment. With the addition of 20 ng/ml rhCTGF C-terminal fragment, the extent of RPE cell migration was similar to that observed with PDGF. Additionally, the chemotactic response observed in RPE cells at 20 ng/ml C-terminal fragment of rhCTGF was essentially equivalent to that seen using 30 ng/ml rhCTGF (see this Example above, and FIG. 5A). Only a minor increase in cell migration above control was observed in RPE cells treating with N-terminal fragment of CTGF. (FIG. 6B.)

Together, these results showed that CTGF and fragments of CTGF stimulated ocular cell chemotaxis and migration. Therefore, CTGF and fragments of CTGF provide novel therapeutic targets for inhibiting ocular cell migration associated with various ocular disorders.

### Example 7

### CTGF Stimulates RPE Cell Proliferation

Under normal conditions in the healthy eye, RPE cells are quiescent, and therefore do not proliferate. In many ocular disorders, however, ocular cell proliferation, including RPE cell proliferation, is observed and associated with development and progression of various ocular disorders. Therefore, the effect of CTGF on ocular cell proliferation was examined as follows.

RPE cell proliferation was measured using a ³H-thymidine uptake/incorporation assay. Human RPE cells were isolated as described above in Example 1 and incubated in 24-well tissue culture plates for 48 hours in DMEM containing 1% fetal bovine serum, in the presence or absence of various concentrations of rhCTGF (1 to 100 ng/ml). Afterward, the RPE cell cultures were pulse-labeled with ³H-thymidine (1 μCi/well, Amersham) for the final 16 hours of incubation. The amount of ³H-thymidine uptake in the cultures was then determined as previously described. (He et al., supra.)

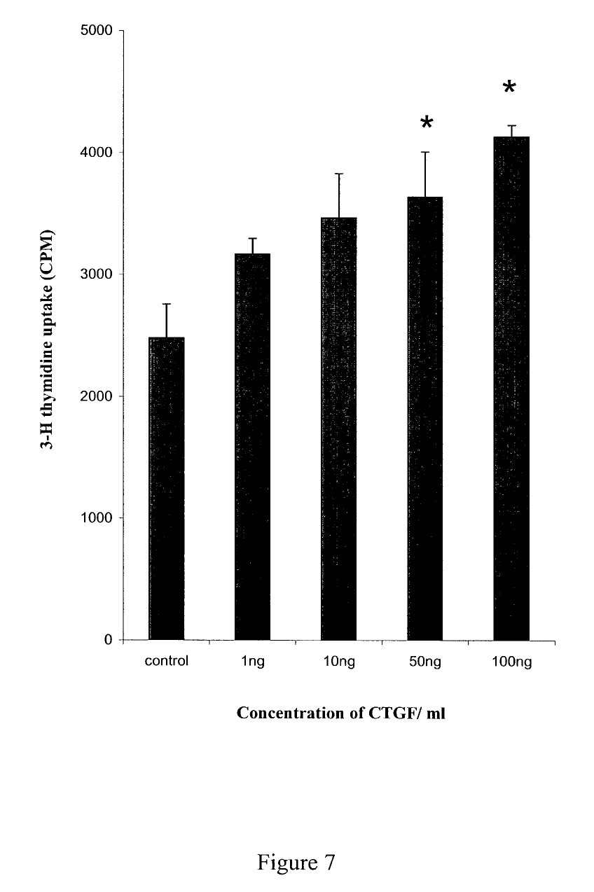

FIG. 7 shows rhCTGF displayed mitogenic activity to RPE cells. Specifically, rhCTGF stimulated a dose-dependent increase in ³H-thymidine incorporation in RPE cells after 48 hours of rhCTGF treatment. At rhCTGF concentrations of 50 ng/ml and 100 ng/ml, rhCTGF stimulated an approximately 20% increase in ³H-thymidine uptake in RPE cells, a statistically significant increase above that of control (indicated by asterisks (P<0.05) in FIG. 7). These results demonstrated RPE cell proliferation was induced by CTGF. Therefore, CTGF stimulates proliferation of ocular cells.

### Example 8

### CTGF Induction of CEC Tube Formation

Angiogenesis and neovascularization involve a complex and highly orchestrated series of cellular events and changes in endothelial cell function, including proliferation, migration, and differentiation. To assay the effect of CTGF on endothelial cell function and neovascularization in the eye, the following tube formation assay was performed on CECs. The tube formation assay is a complex three-dimensional assay that examines the ability of endothelial cells to form primitive vascular structures or vascular tubes in culture. For primitive vascular tubes to form using this assay, endothelial cells undergo proliferation, migration, differentiation, and organization into three-dimensional structures. Therefore, the effect of CTGF on CEC tube formation was examined using this assay.

Bovine CECs were isolated as described above in Example 1. Monolayer cultures of CECs were grown on fibronectin-coated 6-well plates. Growth media was removed and replaced with fresh EGM medium containing 1% fetal bovine serum. Recombinant human CTGF (20 ng/ml) was added to the cultures and the CECs were incubated for an additional 9 days. Tube formation was monitored and photographed daily using phase-contrast microscopy.

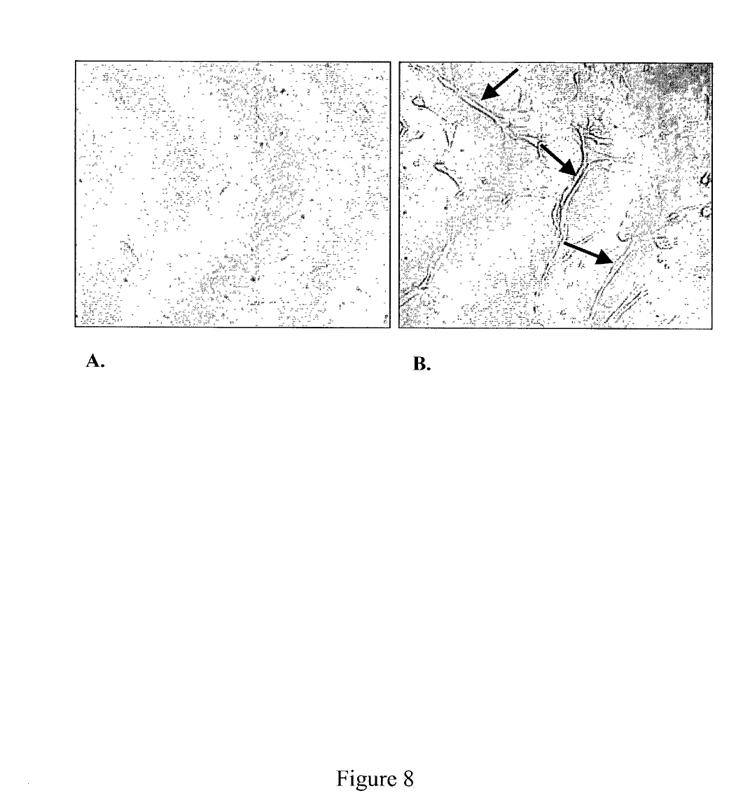

Monolayer cultures of CECs showed increased tube formation in response to rhCTGF addition. As shown in FIG. 8A, in the absence of rhCTGF addition, CECs remained as monolayer cultures. As shown in FIG. 8B, in the presence of rhCTGF, the attached endothelial cells became extended, formed cell-cell contacts, and established a branched network of tube-like structures. (See arrows, FIG. 8B.) These results were similar to those previously reported, showing the effect of VEGF (a known angiogenic factor) on endothelial cell tube formation. (Murata et al. (2000) Invest Ophthalmol Vis Sci 41:2309-2317.) Therefore, these results demonstrated rhCTGF induced tube formation in cultured CECs, indicating CTGF stimulated ocular endothelial cell differentiation, proliferation, and migration. Additionally, these results suggested CTGF could induce neovascularization in the eye, including neovascularization associated with various ocular disorders.

### Example 9

### CTGF Induces Expression of Extra Domain A Fibronectin in RPE Cells

Increased expression of extra domain A fibronectin (EDA(+)FN) is associated with tissue injury, including ocular tissue injury. Specifically, cellular fibronectin expression has been identified in tissue samples obtained from patients with PVR and CNV. To examine the effect of CTGF on cellular fibronectin expression in ocular cells, the following experiments were performed.

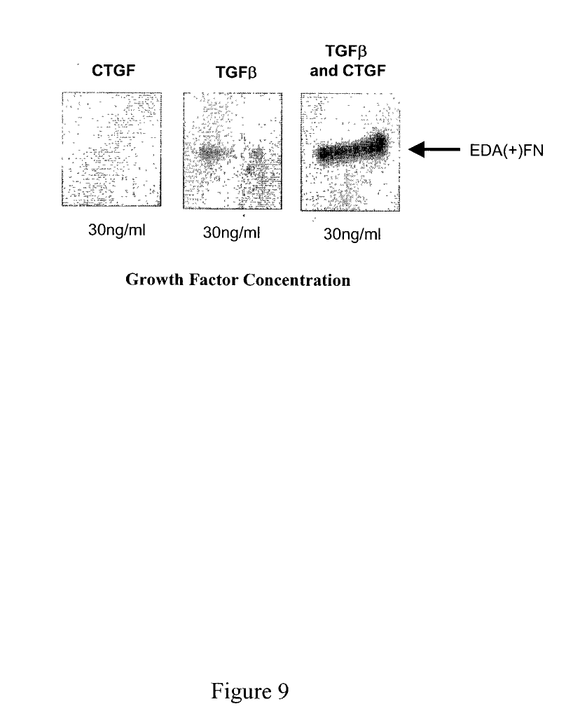

Cultures of human fetal RPE cells (passages 2 to 4) were grown to subconfluence as described in Example 1. RPE cells were serum starved for 24 hours followed by treatment with rhCTGF (30 ng/ml), TGFβ2 (30 ng/ml), or both growth factors, for 48 hours in serum-free medium. Afterward, RPE cells were collected and lysed using lysis buffer. The resulting cellular homogenates were passed through a 25G needle to sheer DNA, incubated on ice for 30 minutes, and then centrifuged at 12,000 rpm for 10 minutes at 4° C. Samples were examined for cellular fibronectin expression (EDA(+)FN isoform) by western blot analysis using a FN EDA-specific monoclonal antibody (Clone IST-9; Accurate Chemicals, Westbury, N.Y.). As shown in FIG. 9, addition of rhCTGF alone resulted in no detectable EDA(+)FN expression in RPE cells. Addition of TGFβ2 alone resulted in weak EDA(+)FN expression in RPE cells. When both CTGF and TGFβ2 were added to cultured RPE cells, a robust increase in EDA(+)FN expression was observed. These results demonstrated that CTGF enhances the ability of TGFβ to stimulate extracellular matrix production in ocular cells.

### Example 10

### Fibronectin Induces Expression of CTGF in RPE Cells

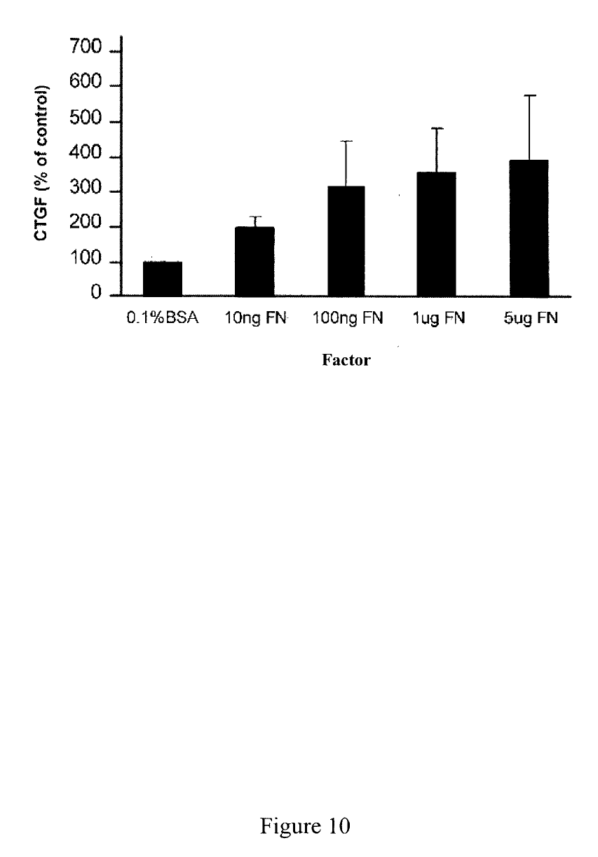

Fibronectin is abundantly expressed in PVR and conditions associated with retinal or choroidal neovascularization. To examine the effects of CTGF on fibronectin expression in ocular cells, the following experiment was performed. RPE cells, isolated as described above in Example 1, were seeded on plastic in 6-well plates pre-coated with various amounts of fibronectin (10 ng to 5 μg). After 24 hours, supernatants were tested for CTGF expression using an ELISA. Cellular homogenates were tested for CTGF expression using western blot analysis. As shown in FIG. 10, RPE cells plated on fibronectin showed a dose-dependent increase in CTGF expression, as measured by densitometric analysis of the western blot analysis of cell homogenates. RPE cells plated on as little as 10 ng fibronectin demonstrated increased CTGF expression above that observed in control RPE cells plated on 0.1% BSA. Fibronectin induced an 80% increase above control in CTGF expression in cellular homogenates of RPE cells plated on fibronectin. ELISA analysis revealed a doubling of the level of both CTGF and N-terminal fragments of CTGF in the supernatants of these cells (data not shown). These results provided evidence that fibronectin stimulates CTGF expression in RPE cells as RPE cells migrate into various ocular environrments containing fibronectin, which occurs in various ocular disorders.

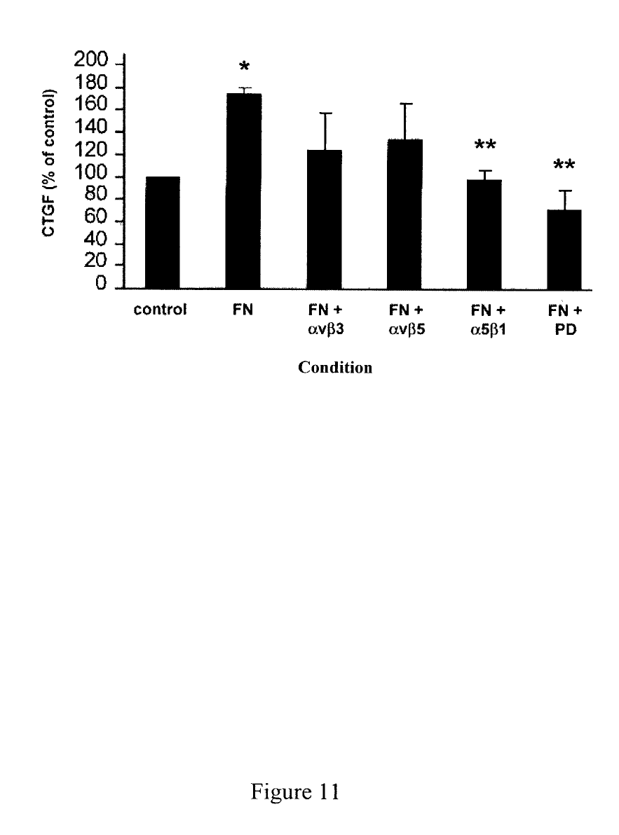

In parallel experiments, pretreatment of cells with neutralizing antibodies directed against α₅β₁ integrin, A fibronectin-specific integrin, significantly blocked the increase in CTGF expression observed in both cell homogenates and supernatants. (FIG. 11.) Addition of antibodies directed against αᵥβ₃ integrin and αᵥβ₁ integrin had no significant effect on fibronectin-induced CTGF expression. These results further supported a role of fibronectin in stimulating CTGF production in ocular cells, as α₅β₁ integrin is associated with attachment of RPE cells to fibronectin.

### Example 11

### CTGF Expression and Its Effect on Retinal Müller Cell Migration

Rat retinal Muller cells were obtained from an established cell line (Sarthy et al. (1998) Invest Ophthalmol Vis Sci 39(1):212-6), and were cultured in the same medium as the RPE cell culture described in Example 1. Expression of CTGF mRNA was examined in cultured retinal Müller cells using RT-PCR. The methods for isolation mRNA and cDNA synthesis from Müller cell were the same as RPE (see example one for detail). PCR amplification was performed for detection of CTGF mRNA expression in the cell using oligonucleotide sequence primer specific for rat CTGF. The primer sequences for rat CTGF were 5′-ctccagtctgcagaaggtattc-3′ (SEQ ID NO.:7) and 5′-caaccgcaagattggagtgt-3′ (SEQ ID NO.:8), which resulted in amplification of a 480 base pair rat CTGF DNA fragment. The conditions used for PCR amplification were as follows: 94° C. for 1 minute; 60° C. for 1 minute; and extension at 72° C. for 2 minutes, total for 35 cycles. The PCR product of rat retinal Müller cell CTGF was resolved in an agarose gel. A PCR amplification product of the expected size was observed (data not shown), indicating rat retinal Muller cells express CTGF.

Müller cell migration was assayed by using a modified Boyden chamber assay, as describd above in Example 6. CTGF induced Müller cell migration in dose dependent manner. At a dose of 30 ng/ml and 50 ng/ml, rhCTGF stimulated a significant increase of migration of Müller cells on fibronectin (data not shown). These results showed CTGF was a chemotactic for Muller cells. These results indicated a role of CTGF on Muller cell migration in development of PVR, as Muller cells are present in PVR membrane.

### Example 12

### CTGF Expression in Human PVR Membrane Tissue

The expression of CTGF in human PVR membranes obtained from individuals with ocular disorders was examined by immunohistochemical analysis. PVR membranes, formed as a result of trauma to the eye or retinal detachment, were surgically removed from patients. The isolated PVR membranes were prepared for immunostaining as follows. Isolated PVR membranes were snap-frozen and sectioned at 6 μm using a cryostat. Thawed tissue sections were air-dried, rehydrated with PBS (pH 7.4), and blocked with 5% normal goat serum for 15 minutes. Sections were incubated with rabbit anti-CTGF polyclonal antibody (primary antibody, prepared using standard methods known in the art) for 60 minutes. Sections were then washed three times with PBS, 5 minutes each wash, to remove unbound anti-CTGF antibody. Sections were subsequently incubated with biotinylated anti-rabbit antibody (secondary detection antibody, diluted 1:400, Vector, Burlingame, Calif.), and washed for 5 minutes with PBS. Streptavidin peroxidase was added, and color development performed using AEC kit (Zymed). Slides were counter-stained with hematoxylin and mounted with glycerin-gelatin medium.

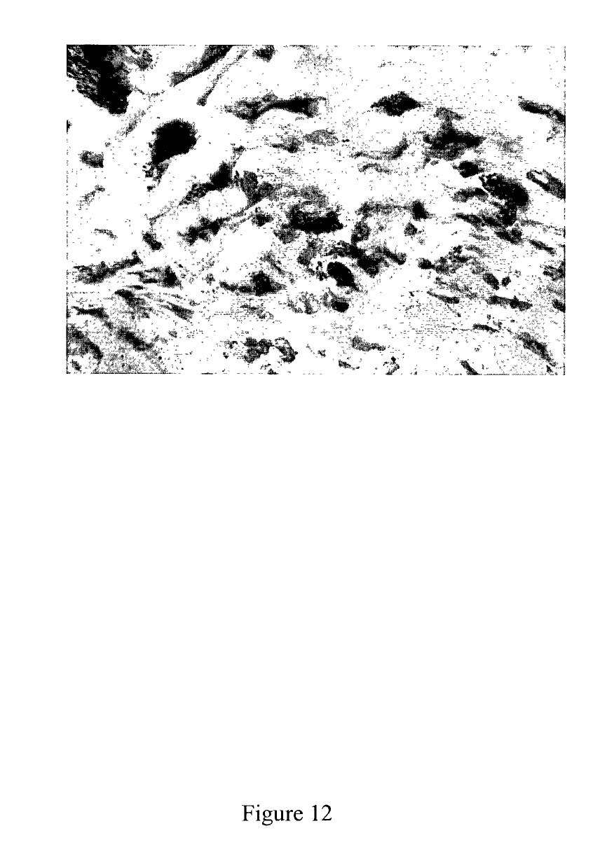

All ten isolated PVR membranes examined stained positive for CTGF expression, as evidenced by immunohistochemistry. Within PVR membranes, CTGF was localized to the extracellular space or to cells within the fibrotic PVR membranes, as shown in FIG. 12. Immunostaining for CTGF was not detected in sections of normal, non-diseased RPE (data not shown). These results showed CTGF was expressed in ocular disorders.

Double immunoperoxidase and alkaline peroxidase staining was also performed on sections of isolated human PVR membranes. Thawed cryosections were fixed for 10 minutes with 10% neutral buffered formalin (Polyscience, Inc., Warrington, Pa.), washed three times (5 minutes each wash) with Tris-HCI (pH 7.4), and blocked with 5% goat serum for 15 minutes. After the sections were immunostained for CTGF using an anti-CTGF antibody, as described above, the slides were washed three times with Tris-HCl buffer. Primary antibody specific for cytokeratin (in the retina, cytokeratin is a specific marker for RPE cells), and primary antibody specific for CD-31 (an endothelial cell-specific marker) were added, and the slides were incubated for 1 hour at room temperature. The slides were washed three times with Tris-HCl buffer. The sections were incubated with alkaline phosphatase-conjugated anti-mouse antibody (Vector, diluted 1:500) for 30 minutes. The slides were again washed three times with Tris-HCl buffer. Vector Blue was then added to the slides for 15 minutes for color development.

CTGF was expressed in isolated human PVR membranes, as evidenced by immunohistochemical analysis. (FIG. 12.) Double-labeling of isolated human PVR membrane sections revealed that many CTGF-positive cells also stained positive for cytokeratin, an RPE cell-specific marker in the retina. These results showed that RPE cells within PVR membranes expressed CTGF.

### Example 13

### CTGF Expression in CNV Membranes

The expression of CTGF in CNV membranes was examined by immunohistochemical staining. CNV membranes were surgically excised from patients with clinically diagnosed age-related macular degeneration. The isolated CNV membranes were prepared for analysis of CTGF expression by immunostaining using methods described in Example 12 above for CTGF immunostaining of PVR membranes.

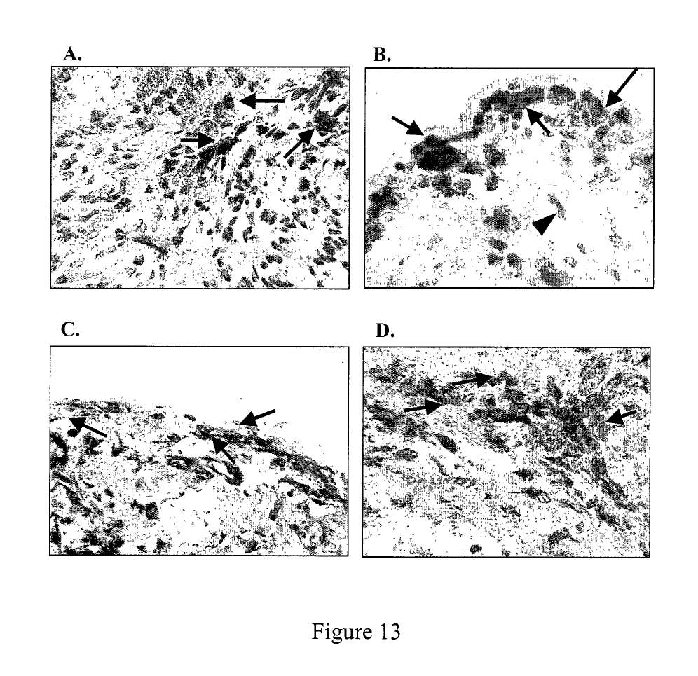

CNV membranes examined stained positive for CTGF expression. The most prominent CTGF staining occurred in vascularized regions of the membrane, as indicated with arrows in FIG. 13A. FIG. 13A shows cytoplasmic staining of CTGF-positive cells (indicated by arrows). The nuclei of cells are stained with hemotoxylin. In various ocular disorders, certain membranes show active neovascularization, while others display fibrous scars with little neovascularization. The results indicated that in CNV, CTGF was predominantly localized to neovascular membranes; however, some CTGF staining was also found in non-neovascular membranes. The intensity and extent of CTGF staining within CNV membranes was several fold greater in vascular areas compared to that observed in non-vascular membranes and fibrotic membranes. Additionally, CTGF-positive cells were found surrounding blood vessels in CNV membranes.

A partially intact RPE monolayer was observed in certain isolated CNV membranes. As indicated by arrows in FIG. 13B, the regions associated with an RPE monolayer stained strongly positive for CTGF. Double staining of the CTGF-positive cells within the intact RPE monolayer demonstrated that many CTGF-positive cells were also positive for cytokeratin immunoreactivity, indicated by arrows in FIG. 13C. The arrowhead shown in FIG. 13B shows a cell migrating form the RPE monolayer stained positive for CTGF. These results indicated that the CTGF-positive cells observed in isolated CNV membranes swere transdifferentiated RPE cells, displaying a change in morphology from that of a typical retinal pigment epithelial cell to a more fibroblast-like appearance. Arrows in FIG. 13D show other CTGF-positive cells in CVN membranes included endothelial cells, as evidenced by double-labeling with the endothelial cell marker CD-31 and CTGF. Normal adult retinas were also examined for CTGF expression; however, no CTGF immunoreactivity was detected in RPE or CEC of these samples (date not shown).

Taken together, these results demonstrated that CTGF is expressed in CNV membranes associated with ocular disorders. CTGF expression in CNV membranes was most prominent in vascular regions of the membranes. Expression of CTGF was localized to stromal RPE and CEC as well as RPE in the residual monolayer. The presence of CTGF in the residual RPE monolayer suggested that CTGF could activate the normal underlying endothelium, leading to stimulation of choroidal angiogenesis and neovascularization, in part, by regulating CEC function.

### Example 14

### CTGF Expression in PVR

The expression of CTGF and fragments of CTGF in vitreous of patients clinically diagnosed with PVR was investigated. Vitreous samples were obtained from different patients with various ocular disorders, including retinal detachment with PVR (RD+PVR), retinal detachment without PVR (RD), epiretinal membrane (ERM), and macular hole (MH), which is not associated with PVR. ELISA assays specific for detecting CTGF and fragments of CTGF were used to determine the levels of CTGF and of fragments of CTGF in these vitreous fluids.

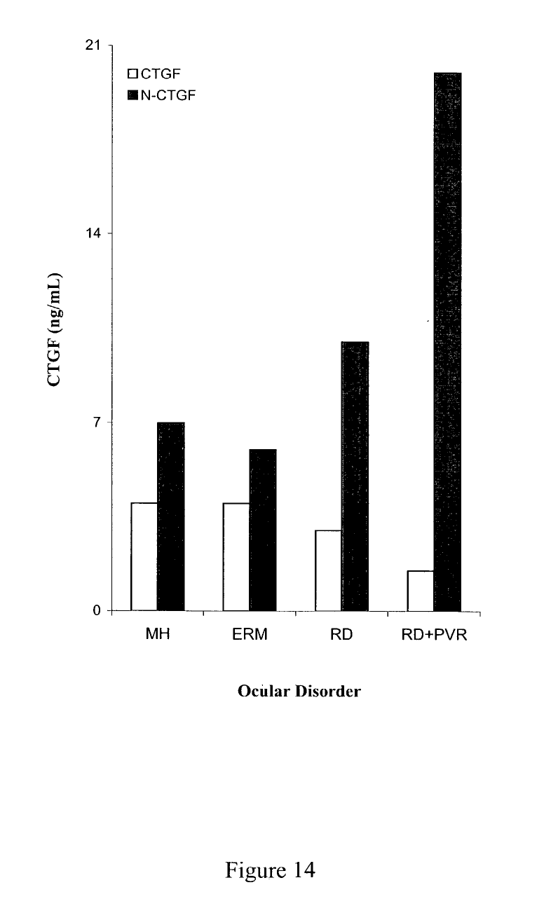

As shown in FIG. 14, CTGF (open bars) and N-terminal fragments of CTGF (solid bars) were detected in vitreous fluid obtained from patients with various ocular disorders, including MH, ERM, RD, and RD+PVR. In all samples tested, detectable levels of N-terminal fragments of CTGF were higher than that of CTGF. A significantly higher level of N-terminal fragments of CTGF was observed in vitreous obtained from patients with PVR compared to that seen in vitreous obtained from patients with eye disease without PVR, such as MH. (FIG. 14.) Specifically, the levels of N-terminal fragments of CTGF in vitreous of patients with RD was approximately 10 ng/ml, while the levels of N-terminal fragments of CTGF in vitreous of patients with RD complicated with PVR was approximately 20 ng/ml. (FIG. 14.) Therefore, RD alone was not associated with a significant increase in the levels of N-terminal fragments of CTGF. However, in RD associated with PVR, N-terminal fragments of CTGF were elevated, suggesting a role of CTGF in the development of PVR in RD.

These results demonstrated that levels of CTGF and fragments of CTGF could be measured quantitatively from cell-free vitreous samples. These results also showed that CTGF levels are greatly elevated in vitreous of patients with ocular disease associated with fibrosis (fibrotic), such as PVR, and, to a lesser extent, in ocular disorders not having a fibrotic aspect, such as MH.

### Example 15

### CTGF Expression in CNV and Retinal Neovascularization

The expression of CTGF in vitreous of patients clinically diagnosed with various forms of ocular neovascularization was investigated. Vitreous samples were obtained from patients with clinically diagnosed CNV and retinal neovascularization (RNV). Samples representing CNV included age-related macular degeneration (AMD), and CNV without AMD (CNVM non-AMD). Samples representing RNV included proliferative diabetic retinopathy (PDR), PDR with vitreous hemorrhage (PDR with VH), and VH without PDR (VH non-PDR). ELISA assays specific for detecting CTGF and fragments of CTGF were used to determine the levels of CTGF and of fragments of CTGF in these vitreous fluids.

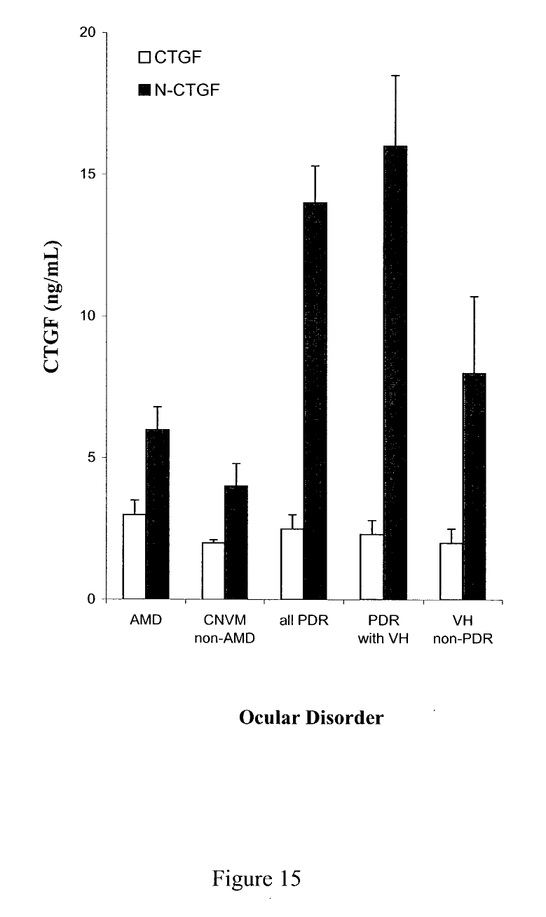

As shown in FIG. 15, CTGF (open bars) and N-terminal fragments of CTGF (solid bars) were detected in vitreous fluid obtained from patients with various ocular disorders, including AMD, CNVM non-AMD, PDR, PDR with VH, and VH non-PDR. In all samples tested, detectable levels of N-terminal fragments of CTGF were higher than that of CTGF. CTGF N-terminal fragment levels were highest in vitreous samples obtained from patients diagnosed with PDR. These results showed that CTGF levels are greatly elevated in vitreous of patients with PDR. Additionally, these results indicated that CTGF expression is associated with ocular disorders associated with neovascularization and ocular cell proliferation.

### Example 16

### Animal Model of PVR Involving RPE Cell Injection with PDGF

Experiments using animal models (pigmented rabbits) of ocular disease were performed. In these animal models of ocular disease, one eye (typically the right eye), is used experimentally from each animal, allowing the other eye to serve as a non-treatment control. Due to the inherent variability of in vivo animal model experiments, each experimental group typically involved 6 rabbits, allowing for the generation of sufficient numbers to achieve statistically valid results. In this established rabbit model of PVR, isolated RPE cells and PDGF are injected into the rabbit eye. In this model, 94% of eyes injected with RPE and PDGF developed retinal focal traction within one week following the injection, and 92% of the injected eyes developed retinal detachment within two weeks of injection.

Rabbit RPE cells were isolated from adult rabbit eyes (rabbits used in the studies weighed 2.5 kg to 3.5 kg.), and cultured in DMEM supplemented with 10% fetal bovine serum. Subconfluent cultures of rabbit RPE cells (typically at passage 2 to 3) were used for all subsequent injections. Cultured rabbit RPE cells were removed from culture plates by trypsinization and resuspended in PBS (250,000 cells per 0.1 ml). Prior to injection of RPE cells into rabbit eyes, 0.2 mls of aqueous humor was removed from each eye using a 25-guage needle. With indirect opthalmoscopic control, a 27-guage needle attached to a tuberculin syringe containing the RPE cells was passed through the sclera at a point three millimeters posterior to the limbus, at a position just over and above the optic disc. Following injection of RPE cells, 150 ng of PDGF-BB in 0.1 ml of PBS was injected through the same entrance site using the same technique as described for injection of RPE cells. Following the injections, animals were examined over time using indirect ophthalmoscopic examinations. The development and extent of PVR was monitored over time, and classified into one of five stages, based on clinical findings, according to parameters described by Fastenberg. (Fastenberg et al. (1982) Am J Ophthalmol 93:565-572.) The stages of PVR development in this rabbit model are outlined in Table 1 below.

TABLE 1
Stages of PVR in the rabbit eye

| | |
|---|---|
| Stage 0 | Normal Fundus |
| Stage 1 | Intravitreal membrane |
| Stage 2 | Focal traction |
| | Localized vascular change |
| | Hypermia, engorgement, dilation, tortuosity, |
| | vessel elevation |
| Stage 3 | Localized detachment of medullary ray |
| Stage 4 | Total ray detachment |
| | Peripapillary detachment |
| Stage 5 | Total retinal detachment |
| | Retinal holes |
| | Retinal folds |

Histological examination of the affected eyes in this animal model of PVR showed the presence of moderately cellular membranes with moderate fibrosis. When the number of RPE cells injected was reduced from 250,000 cells, as described above, to 100,000 cells, the resulting PVR was mild, and only fragile, paucicellular, non-fibrotic membranes developed. Additionally, membranes that developed in eyes injected with reduced numbers of RPE cells (i.e., 100,000 cells) were focal and localized, no retinal detachment was observed.

### Example 17

### CTGF Induces PVR in Animal Model of PVR

The effect of CTGF on the development of PVR was investigated, using a modification of the rabbit model of PVR described above in Example 16. Initially, various amounts of rhCTGF were injected into the rabbit eye. The effect of intra-vitreous rhCTGF injection alone on RPE, and any resulting development of PVR, was examined. As indicated in Table 2, vitreous injection of either 200 ng or 400 ng of rhCTGF into a healthy rabbit eye had no detectable effect on RPE. Subretinal injection of 200 ng of rhCTGF, however, resulted in minor RPE changes, including slight, focal disorganization of the RPE monolayer. No significant cell proliferation or membrane formation was observed. (See Table 2 below.)

TABLE 2
Intravitreous injection of rhCTGF into the rabbit eye

| Injection Site | Amount of rhCTGF Injected | Effect/Outcome |
|---|---|---|
| Vitreous injection | 200 ng CTGF | No effect on RPE |
| | 400 ng CTGF | No effect on RPE |
| Subretinal injection | 200 ng CTGF | Minor RPE changes |

In the rabbit model of PVR described above in Example 16, RPE cells injected into the rabbit eye, along with PDGF, resulted in development of a mild degree of PVR within two weeks of injection, characterized by formation of cellular membranes with moderate fibrosis. To examine the effects of CTGF in this rabbit model of mild PVR, RPE cells were injected into the rabbit eye, as described above, along with various amounts of rhCTGF, without PDGF injection. As shown in Table 3 below, when RPE cells were injected into the rabbit eye along with 200 ng,rhCTGF, stage I PVR resulted after 3 weeks. Histological examination of the injected eye did not reveal the presence of epiretinal membranes or retinal detachment.

When RPE cells were injected into the rabbit eye along with 400 ng rhCTGF, grade 2 PVR developed after 3 weeks. Small and thin membranes developed, as determined by histological examination of the injected eye. The results of injecting various amounts of rhCTGF together with RPE cells into the rabbit eye are summarized in Table 3 below. Taken together, these results indicated that rhCTGF, when injected intravitreously, had a minimal effect on RPE.

TABLE 3
Intravitreous injection of RPE cells and rhCTGF into the rabbit eye

| | Amount of | RPE Stage | |
|---|---|---|---|
| RPE Cells Injected | rhCTGF Injected | (after 3 weeks) | Histology |
| 100,000 RPE cells | No CTGF | Stage 0 | Negative |
| 100,000 RPE cells | 100 ng CTGF | Stage 0 | Negative |
| 100,000 RPE cells | 200 ng CTGF | Stage 1 | Negative |
| 100,000 RPE cells | 400 ng CTGF | Stage 2 | Small and thin |
| | | | membranes |

A positive control for the development of PVR in this animal model was intravitreous injection of 100,000 RPE cells with 50 ng PDGF. Three weeks following such injection, tractional retinal detachment was observed in the injected eye, associated with thin paucicellular membranes. (See Table 4 below.) In a parallel experiment, one week following the injection of 100,000 RPE cells with 50 ng PDGF, 200 ng rhCTGF was injected intravitreously. Two weeks later, eyes were examined for any changes. Under these experimental conditions, thick, fibrotic membranes developed, representing PVR stage 3. These results showed that when CTGF was injected into the vitreous of a rabbit having a mild degree of PVR, the membranes became densely fibrotic. Therefore, in animal models of mild PVR, intravitreous CTGF addition stimulated and enhanced the development of intraocular fibrosis. These results also showed direct evidence that CTGF induced pathologic fibrosis in vivo in an animal model of ocular disease. These results also showed that CTGF induces ocular cell proliferation and acts, at least in part, in the context of other growth factors associated with ocular disorders to induce ocular fibrosis.

TABLE 4
CTGF enhances PDGF-induced PVR in rabbit model

| RPE Cell | PDGF | rhCTGF | RPE | |
|---|---|---|---|---|
| Injected | Injected | Injected | Stage | Histology |
| 100,000 RPE | 50 ng PDGF | No CTGF | Stage 3 | Retinal |
| cells | | | | detachment |
| | | | | Thin paucicellular |
| | | | | membranes |
| 100,000 RPE | 50 ng PDGF | 200 ng CTGF¹ | Stage 5 | Thick, fibrotic |
| cells | | | | membranes |

¹rhCTGF injected 1 week after RPE cell and PDGF injection

### Example 18

### Adenovirus-CTGF Expression Vector Construction

Adenovirus expression vectors were designed and constructed for adenovirus-mediated expression of CTGF and fragments of CTGF for use in various in vivo and in vitro experiments. A CTGF-containing adenovirus expression vector construct (pAd-CTGF) was prepared using the AdEasy system, commercially available from Qbiogene (Montreal, Canada). Components of the system include pAdEasy-1 (the viral backbone, Catalog No. AES1010) and pShuttle-CMV (Catalog No. AES1021). A full-length human CTGF cDNA (DB60R32, GenBank Accession No. M92934, GI: 180923) was obtained from Dr. Gary Grotendorst (University of Miami Medical School). (Bradham et al. (1991) J Cell Biol 114:1285-1294.) CTGF cDNA was excised from pBluescript II KS (DB60R32) with the restriction enzymes BamHI (which cleaves pBluescript II KS at position 689) and SwaI (which cleaves CTGF cDNA at a position 1277 bp downstream (i.e., located 3′) to the 5′ end. The CTGF cDNA fragment was then inserted into pShuttle-CMV, previously digested with the restriction enzymes BglII and EcoRV, using standard molecular cloning techniques. CTGF expression from the resulting pShuttle-CMV-CTGF construct was confirmed by transient expression of and western blot analysis of expressed proteins, using standard methods.

The pShuttle-CMV-CTGF expression plasmid was used to generate a recombinant adenovirus by bacterial recombinantion with pAdEasy-1 by following the manufacturer's procedures. Briefly, pShuttle-CMV-CTGF was linearized then co-transfected (by electorporation) with pAdEasy-1 into competent E. coli BJ-5183 cells. The resulting clones were cultured in LB-KM medium for 16 hr and the recombinant pAd-CTGF plasmid was extracted by alkaline extraction methods. The control plasmids pAd-GFP (containing green fluorescent protein) and pAd-e1E3del.pSCMV (control adenovirus plasmid containing no insert) were also prepared. The recombinant plasmids sequences were confirmed by restriction enzyme mapping using several restriction enzymes. Automated DNA sequencing using an ABI prism 310 automated DNA sequencer further confirmed the CTGF cDNA sequence in pAd-CTGF. Isolated plasmids were transformed to stable E. coli JM 109 cells. Recombinant plasmids were subsequently linearized with the restriction enzyme Pac1, and transfected with Lipofectamine (GIBCO/BRL, Grand Island, N.Y.) into 293 packaging cells containing E1 and E3 genes. After transfection, the cells were collected and subject to three rounds of freeze-thaw. The supernatants were collected. Viral particles were purified by CsCl gradient ultracentrifuge using 1.30 gravity CsCl solution in 10 mM Tris-HCl, pH 7.4. The titer of the virus was determined by PFU assay.

A replication-deficient recombinant adenovirus gene containing a cDNA fragment encoding human CTGF N-terminal fragments or C-terminal fragments is constructed as follows. Poly(A)⁺ RNA is isolated from cultured human RPE cells using a FastTrack Kit (Invitrogen, San Diego, Calif.), according to methods described by the manufacturer. First strand cDNA is synthesized at 42° C. with M-MLV reverse transcriptase (Gibco/BRL). PCR reaction is performed using oligonucleotide sequence primers specific for human N-terminal CTGF and C-terminal CTGF, and can be selected by standard methods. The use of these primers results in a CTGF-specific DNA amplification product encoding an N-terminal fragment of human CTGF and a C-terminal fragment of CTGF.

PCR amplification is performed using a Perkin-Elmer/Centur DNA thermal cycler, using, fro example, the following conditions: denaturation at 94° C. for 1 minute; annealing at 60° C. for 1 minute; and extension at 72° C. for 45 seconds. Thirty cycles of PCR are performed. The resulting amplification product is isolated and subcloned into the vector pCR 2.1 (Invitrogen Corp.). The DNA sequences of the CTGF N-terminal and C-terminal fragment cDNAs are confirmed by sequencing, using an ABI prism 310-auto sequencer (ABI, USA). The CTGF N-terminal fragment cDNA and C-terminal fragment cDNA is excised from the pCR 2.1 vector construct by digestion using the restriction enzymes Xbal and Xho1. The resulting restriction fragment is isolated and subcloned into the XbaI-XhoI cloning site of pAdTrack-CMV shuttle vector (ADENO-QUEST, Quantum Biolab Inc., Montreal, Canada). pAdTrack-CMV is a shuttle plasmid useful for homologous recombination, and includes the following elements: adenovirus type 5, E. coli duplication origin, kanamycin resistance gene, eGFP gene (enhanced green fluorescent protein (Clontech, Palo Alto, Calif.) whose expression is driven by human cytomegalovirus immediate-early promoter/enhancer, and a multiple cloning site into which DNA is subcloned and expressed from the CMV promoter). The pAdTrack-CMV vector, which includes CTGF N-terminal fragment cDNA or CTGF C-terminal fragment cDNA, is linearized by the restriction enzyme Pme1, and subsequently co-transfected with pAdEasy-1 (ADENO-QUEST, Quantum Biolab Inc.) by electroporation into competent E. coli BJ-5183 cells.

The resulting clones were cultured in LB-KM medium for 16 hr and the recombinant plasmids (pAdEasy-CTGF-N, pAdEasy-GFP) were extracted by alkaline extraction methods. The control vector pAdEasy-GFP was constructed using the method described above, excluding insertion of CTGF cDNA. The recombinant plasmid sequences were confirmed by restriction enzyme mapping using several restriction enzymes (e.g., EcoR1, Xho1, Sal1, BamH1, and Pac1). Isolated plasmids were transformed to stable E. coli JM109 cells. Recombinant plasmids were subsequently linearized with the restriction enzyme Pac1, and transfected with Lipofectamine (GIBCO/BRL, Grand Island, N.Y.) into 293 packaging cells containing E1 and E3 genes. The sequence of the CTGF cDNA in the vector was confirmed by automated DNA sequencing using an ABI prism 310 auto sequencer. After transfection, the cells were collected, frozen, and thawed three times. The supernatants were collected. Viral particles were purified by CsCl gradient ultracentrifuge using 1.30 gravity CsCl solution in 10 mM Tris-HCl, pH 7.4. The titer of the virus was determined by PFU assay.

### Example 19

### Animal Model of PVR with pAd-CTGF-Infected RPE Cells

As shown in the Examples above, the present invention established that RPE cells express and respond to CTGF, and that RPE cells associated with human PVR membranes are positive for CTGF protein expression. These data indicated a role for CTGF in the initiation, formation, development, and maintenance of PVR and PVR membranes associated with various ocular disorders. In the experiments described above in Example 17, RPE cells were injected into the rabbit eye along with exogenously added rhCTGF. The effect observed was relatively minor (See Table 3), possibly due to lack of sustained CTGF exposure or presence within the ocular environment.

In order to investigate the consequence of continual expression and exposure of CTGF within the eye, as would be observed in developing ocular disorders, the rabbit eye model of PVR described above was modified to provide continual CTGF expression within the eye. The modification included the injection of RPE cells previously infected with adenovirus constructs designed to express rhCTGF, as described below.

Confluent cultures of rabbit RPE cells were washed with serum-free growth media and infected with the 10⁷ PFU of a replication-defective adenovirus vector containing CTGF (Ad-CTGF), constructed as described above in Example 18. After 60 minutes, the medium was replaced with fresh medium containing 2% fetal bovine serum. The infected cells were then incubated for 48-72 hrs at 37° C., after which the cells were removed from the plates by trypsin digestion, washed, and resuspended to 1×10⁷ cells/ml in PBS. Using this procedure, greater than 80% of the cultured RPE cells were infected with Ad-CTGF adenovirus.

Three groups of animals were examined in the following study. Negative control animals were injected with RPE cells that had not been infected with adenovirus. The experimental group of animals was injected RPE cells infected with Ad-CTGF. A third group of animals was injected with RPE cells infected with Ad-GFP, which served as an adenovirus control. Additionally, Ad-GFP-infected cells express green fluorescent protein expression, which allowed monitoring of the fate of injected cells using a green fluorescent protein marker. Prior to injection of RPE cells (either adenovirus-infected or control RPE cells) into pigmented rabbit eyes, the intraocular pressure of the rabbit eye was first reduced by withdrawal of 0.2 ml of aqueous humor using a 23-guage needle. With the use of an operating microscope, a 27-gauge needle connected to a tuberculin syringe containing 100,000 adenovirus-infected RPE cells in 0.1 ml PBS was injected into the vitreous by inserting the needle through the sclera, 3 mm posterior to the limbus.

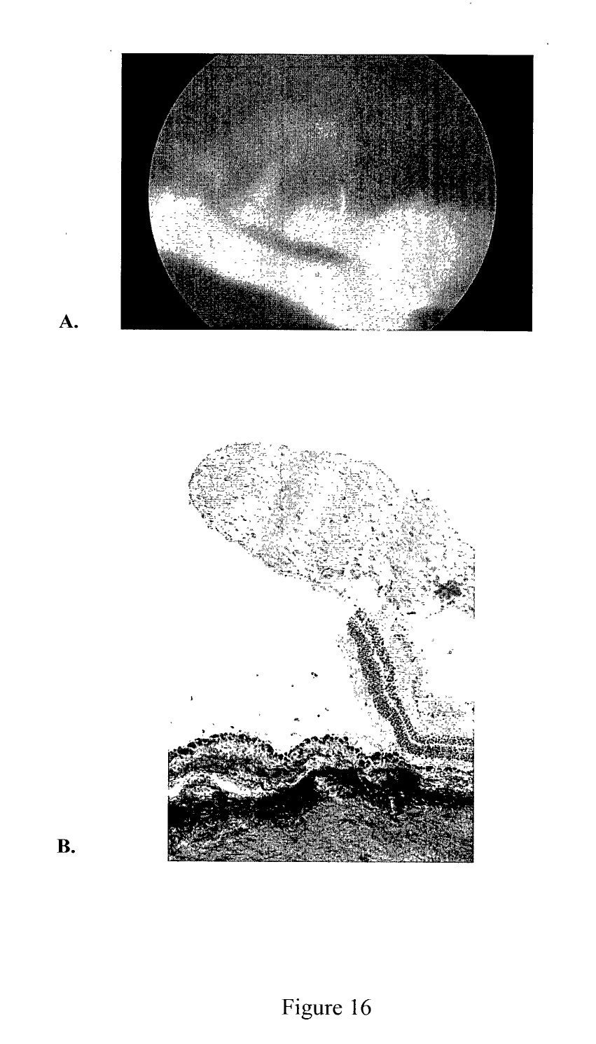

Intravitreous injection of 100,000 RPE cells infected with Ad-CTGF resulted in the formation of a thick membrane, which developed from the optic nerve and extended into the vitreous. (FIGS. 16A and 16B.) Histological analysis showed the presence of cellular and fibrotic epiretinal membranes. Vitreous haze was evident up to 14 days post-injection; the anterior chamber of the eye remained clear.

PVR developed in eyes injected with Ad-CTGF. No retinal break was observed. Evidence of PVR included the appearance of preretinal membranes, distortion of myelinated fibers and retinal vessels, fixed retinal folds, and traction retinal detachment. Additionally, a mass of fibrotic tissue was present in the eye cup. Histological analysis showed a dense fibrotic epiretinal membrane. Neither PVR nor formation of fibrotic epiretinal membranes developed in saline-injected control animals, in animals injected with control adenovirus, or in animals injected with control, non-adenovirus infected RPE cells.

In combination, these results demonstrated that intravitreal injection of RPE cells over-expressing rhCTGF induced ocular fibrosis. Specifically, CTGF addition to an eye in which activated RPE or cellular RPE membranes are present stimulated the formation of intraocular fibrosis, including the transformation of cellular RPE membranes into paucicellular, myofibroblastic, densely fibrotic epiretinal membranes. Additionally, these results indicated that over expression of CTGF initiated the development of PVR without the addition of exogenous growth factors or cytokines typically found in PVR membranes, such as, for example, PDGF, TNFα, TGFβ, HGF, VEGF, IL-1, and IL-6. Therefore, in this animal model of ocular disorder, CTGF provided the initial triggering event leading to fibrosis within a developing non-fribrotic cellular epiretinal membrane. These results showed direct evidence that CTGF induced fibrosis in vivo in an animal model of ocular disease.

### Example 20

### Subretinal—Intravitreous Injection of Adenovirus Model

A pars plana incision is made 3 mm behind the limbus in pigmented rabbits with a stiletto blade. A glass micropipette connected to a micro syringe is inserted under vision control using the operating microscope and contact lens inserted. The retina is gently touched by the micropipette at a distance of 2-3 disc diameters from the optic disc, creating a small retinal hole. Eyes that develope hemorrhage as a result of this procedure are excluded from further study. Two groups of animals are examined in the present study. The experimental group of animals is injected with 50 μl Ad-CTGF. The second group of animals is injected with Ad-GFP, which serves as a control vector and a means to determine which cells were transfected by monitoring expression of green fluorescent protein marker.

### Example 21

### Dispase Model of PVR

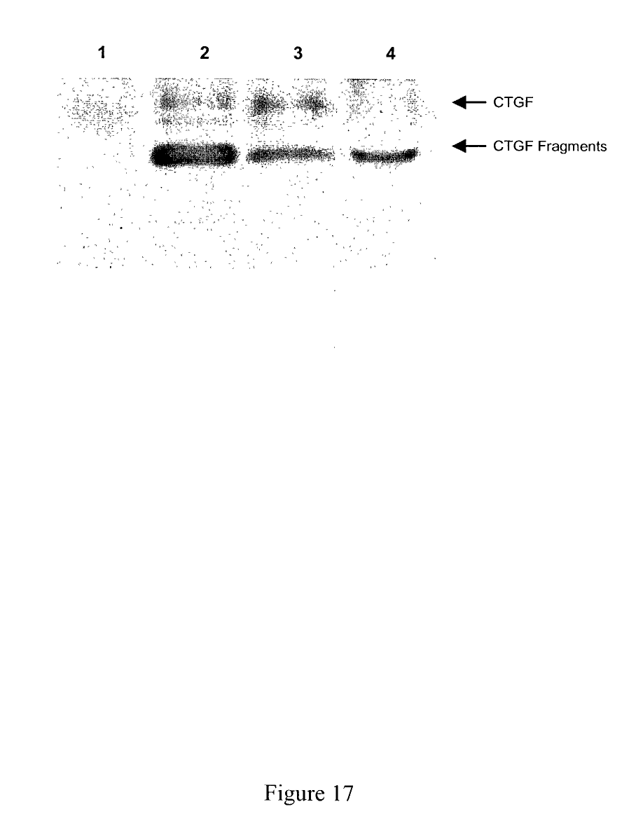

PVR was induced in rabbit eyes using subretinal injection of dispase, using the model and a procedure adapted from that previously described. (Frenzel et al. (1998) Invest Ophthamol Vis Sci 39:2157-2164.) A subretinal bleb was formed as described above using 50 μl (0.05 U) of Dispase (Sigma Chemical Co.) in PBS. Retinal detachment was induced in approximately 75% of the injected rabbits one week after surgery, and in approximately 100% of the injected animals two weeks following surgery. Epiretinal membranes showed moderate fibrosis. Western blot analysis was performed on vitreous aspirates of these animals, as described below in Example 22. As shown in FIG. 17, lane 4, the vitreous of these animals demonstrated increased expression of CTGF and fragments of CTGF above that observed in control conditions (FIG. 17, lane 1).

### Example 22

### Western Blot of Vitreous Aspirates from Animal Models of PVR

Aspirates of rabbit vitreous from various animal models of PVR were examined for CTGF expression by immunoblot analysis. Assays were performed to determine whether CTGF or CTGF fragments accumulated in the vitreous of rabbits with PVR. Results from these experiments are shown in FIG. 17. Positions of CTGF and CTGF fragments are indicated by arrows in FIG. 17. Normal rabbit vitreous (FIG. 17, lane 1) contained low levels of CTGF. PVR was induced by intravitreous injection of rabbit RPE cells previously infected with Ad-CTGF, as described above in Example 19. In these animals, PVR developed initially after 1 week (FIG. 17, lane 3) and became progressively fibrotic after 2 weeks (FIG. 17, lane 2). There is predominant expression of CTGF fragments (approximately 18 kDa) in the vitreous of these animals.

### Example 23

### The Effect of CTGF on the Progression of Gliosis in a Mouse Organ Culture PVR Model

Ocular gliosis is associated with hypertrophy and proliferation of ocular glial cells. Ocular glial cells, including Müller cells and astrocytes, maintain the physical environment and provide structural support to the retina. Ocular gliosis can affect contraction of the retina and can increase the occurrence of retinal detachment.

Eyes from the mouse strain c57B1/6 were used. Eyes were cut at the equator and the cornea and lens removed. The posterior eyecup was placed in 24-well culture plates in Neurobasal medium containing 10% FBS. CTGF (50 ng/ml) was added to the culture medium on the second day of organ culture. Cultures were followed for 7 days for changes including development of retinal folds and rolling up of the retinal edge. Determinations were made by review of photographs and by histology. CTGF stimulated the formation of both retinal folds and rolling of retinal edges when compared with control cultures. Immunohistochemical staining using a glial fibrillary acidic protein (GFAP) antibody revealed that in the areas of pathology, there was a proliferation of GFAP+ glial cells. These results showed that CTGF induced gliosis in perivascular astrocytes. The results obtained with these explant cultures also suggested that CTGF plays an important role in the pathogenesis of PVR by stimulating retinal gliosis and contraction.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for inhibiting or preventing an ocular process associated with connective tissue growth factor (CTGF) in a subject, the method comprising administering an agent that decreases expression or activity of CTGF or fragments thereof.

2. The method of claim 1, wherein the administering is in vivo.

3. The method of claim 1, wherein the administering is in vitro.

4. The method of claim 1, wherein the subject is a cell.

5. The method of claim 1, wherein the subject is an animal.

6. The method of claim 1, wherein the subject is a human.

7. The method of claim 1, wherein the agent is selected from the group consisting of an antibody, an antisense oligonucleotide, and a small molecule.

8. The method of claim 1, wherein the agent is delivered to the eye.

9. The method of claim 1, wherein the agent is delivered to the ocular surface.

10. The method of claim 1, wherein the agent is delivered in an eye drop formulation.

11. The method of claim 1, wherein the ocular process is further associated with an ocular cell.

12. The method of claim 11, wherein the ocular cell is selected from the group consisting of a an endothelial cell, an epithelial cell, a fibroblast, a glial cell, a retinal pigment epithelial cell, a retinal endothelial cell, a choroidal endothelial cell, a lens epithelial cell, a corneal epithelial cell, a trabecular meshwork cell, a pericyte, an astrocyte, a microglial cell, a perivascular glial cell, a Muller cell, a perivascular astrocyte, a rod, a cone, a ganglion cell, and a bipolar cell.

13. The method of claim 1, wherein the ocular process is further associated with an ocular structure.

14. The method of claim 13, wherein the ocular structure is selected from the group consisting of the retina, retinal pigment epithelium layer, choroid, macula, cornea, lens, iris, sclera, trabecular meshwork, aqueous, aqueous chamber, vitreous, ciliary body, optic disc, papilla, and fovea.

15. The method of claim 1, wherein the ocular process is further associated with an ocular disorder.

16. The method of claim 15, wherein the ocular disorder is selected from the group consisting of glaucoma, cataract, choroidal neovascularization, retinal detachment, proliferative vitreoretinopathy, macular degeneration, diabetic retinopathy, corneal-scarring, and corneal haze.

17. The method of claim 1, wherein the ocular process is further associated with ocular fibrosis.

18. A method for inhibiting or preventing ocular extracellular matrix production or deposition, the method comprising administering to a subject an agent that decreases expression or activity of CTGF or fragments thereof.

19. A method for inhibiting or preventing ocular neovascularization, the method comprising administering to a subject an agent that decreases expression or activity of CTGF or fragments thereof.

20. The method of claim 19, wherein the neovascularization is retinal neovascularization, choroidal neovascularization, neovascularization of the iris, or trabecular neovascularization.

21. A method for inhibiting or preventing ocular inflammation, the method comprising administering to a subject an agent that decreases expression or activity of CTGF or fragments thereof.

22. A method for inhibiting or preventing ocular cell proliferation, the method comprising administering to a subject an agent that decreases expression or activity of CTGF or fragments thereof.

23. The method of claim 22, wherein the ocular cell proliferation is selected from the group consisting of epithelial cell proliferation, endothelial cell proliferation, retinal pigment epithelial cell proliferation, and choroidal endothelial cell proliferation.

24. A method for inhibiting or preventing ocular cell migration, the method comprising administering to a subject an agent that decreases expression or activity of CTGF or fragments thereof.

25. The method of claim 24, wherein the ocular cell migration is selected from the group consisting of epithelial cell migration, retinal pigment epithelial cell migration, endothelial cell migration, and choroidal endothelial cell migration.

26. A method for inhibiting or preventing ocular scarring, the method comprising administering to a subject an agent that decreases expression or activity of CTGF or fragments thereof.

27. The method of claim 1, wherein the ocular process is further associated with surgery.

28. The method of claim 27, wherein the surgery is selected from the group consisting of refraction correction surgery, radial keratotomy, LASIK, retinal detachment surgery, corneal transplantation, glaucoma filtration surgery, cataract extraction surgery, lens replacement surgery, vitrectomy, subretinal surgery, and retinal translocation surgery.

29. A method for treating or preventing an ocular disorder, the method comprising administering to a subject an agent that decreases expression or activity of CTGF or fragments thereof.

30. A method for treating or preventing ocular fibrosis, the method comprising administering to a subject an agent that decreases expression or activity of CTGF or fragments thereof.

31. A method for treating or preventing an ocular disorder associated with neovascularization, the method comprising administering to a subject an agent that decreases expression or activity of CTGF or fragments thereof.

32. A method for treating or preventing an ocular disorder associated with extracellular matrix production or deposition, the method comprising administering to a subject an agent that decreases expression or activity of CTGF or fragments thereof.

33. A method for treating or preventing an ocular disorder associated with inflammation, the method comprising administering to a subject an agent that decreases expression or activity of CTGF or fragments thereof.

34. A method for treating or preventing an ocular disorder associated with cell proliferation, the method comprising administering to a subject an agent that decreases expression or activity of CTGF or fragments thereof.

35. A method for treating or preventing an ocular disorder associated with cell migration, the method comprising administering to a subject an agent that decreases expression or activity of CTGF or fragments thereof.

36. A method for treating or preventing a disorder associated with formation or alteration of an ocular membrane, the method comprising administering to a subject an agent that decreases expression or activity of CTGF or fragments thereof.

37. The method of claim 36, wherein the ocular membrane is selected from the group consisting of an epiretinal membrane, a subretinal membrane, a vitreous membrane, a cellular membrane, a choroidal neovascularization membrane, and a fibrotic membrane.

38. A method for maintaining or improving vision in a subject, the method comprising administering to the subject an effective amount of an agent that decreases expression or activity of CTGF or fragments thereof.

39. A method for diagnosing an ocular disorder associated with CTGF, or identifying a predisposition to develop an ocular disorder, the method comprising:
(a) obtaining a sample from a subject;
(b) quantitating the level of CTGF or fragments thereof in the sample; and
(c) comparing the level of CTGF or fragments thereof in the sample to a standard level, wherein an increased amount of CTGF or fragments thereof in the sample is indicative of the presence of an ocular disorder or a predisposition to develop an ocular disorder.

40. A diagnostic kit for use in diagnosing an ocular disorder associated with CTGF, or identifying a predisposition to develop an ocular disorder associated with CTGF, the kit comprising:
(a) a means for detecting the level of CTGF in a sample; and
(b) a means for measuring the level of CTGF in the sample.

41. A diagnostic kit for use in diagnosing ocular fibrosis, or identifying a predisposition to develop ocular fibrosis, the kit comprising:
(a) a means for detecting the level of CTGF in a sample; and
(b) a means for measuring the level of CTGF in the sample.

42. A method for identifying an agent for use in inhibiting or preventing an ocular process associated with CTGF, the method comprising:
(a) contacting a candidate agent with CTGF or a fragment thereof;
(b) detecting the level of CTGF or CTGF fragment expression or activity in the sample; and
(c) comparing the level of CTGF or CTGF fragment expression or activity in the sample to a standard level of CTGF or CTGF fragment expression or activity, wherein decreased expression and activity of CTGF or CTGF fragments in the sample is indicative of an agent that inhibits or prevents an ocular process associated with CTGF.

43. A method for identifying an agent for use in treating or preventing an ocular disorder associated with CTGF, the method comprising:
(a) contacting a candidate agent with CTGF or CTGF fragments;
(b) detecting the level of CTGF or CTGF fragment expression or activity in the sample; and
(c) comparing the level of CTGF or CTGF fragment expression or activity in the sample to a standard level of CTGF or CTGF fragment expression or activity, wherein decreased expression and activity of CTGF or CTGF fragments in the sample is indicative of an agent that treats or prevents an ocular disorder associated with CTGF.
